(19) United States
(12) Patent Application Publication
(10) Pub. No.: US 2019/0119380 A1
(43) Pub. Date: Apr. 25, 2019

(54) FUNCTIONAL PLASTICITY OF ILC2, IMMUNITY, AND COPD

(71) Applicant: MEDIMMUNE, LLC, GAITHERSBURG, MD (US)

(72) Inventors: ALISON A. HUMBLES, GAITHERSBURG, MD (US); JONATHAN S. SILVER, GAITHERSBURG, MD (US); ROLAND KOLBECK, GAITHERSBURG, MD (US)

(21) Appl. No.: 16/094,938

(22) PCT Filed: Apr. 19, 2017
(86) PCT No.: PCT/US17/28369

§ 371 Date: Oct. 19, 2018

Related U.S. Application Data
(60) Provisional application No. 62/326,465, filed on Apr. 22, 2016.

(51) Int. Cl. C07K 16/28; A61K 38/20; A61P 35/04; A61P 11/06; A61K 35/17; C07K 14/54

(52) U.S. Cl. CPC C07K 16/2803; A61K 38/208; C07K 14/54; A61P 11/06; A61K 35/17; A61P 35/04

(57) ABSTRACT

The disclosure provides a method for treating or preventing a disease or disorder associated with lung inflammation by inhibiting the conversion of type 2 innate lymphoid cells (ILC2s) to type 1 innate lymphoid cells (ILC1s).

### BACKGROUND

Innate lymphoid cells (ILCs) are a recently described population of tissue-resident, innate lymphocytes with diverse roles in inflammation, including defense against pathogens, maintenance of epithelial barrier function, containment of commensal microbiota, tissue repair, and regulation of metabolism. (Artis, D. & Spits, H. The biology of innate lymphoid cells. Nature 517, 293-301 (2015); McKenzie, A., Spits, H., & Eberl, G. Innate Lymphoid Cells in Inflammation and Immunity. Immunity 41, 366-374 (2014)). Despite the limited numbers of these cells, they significantly impact ongoing immune responses. ILCs are classified into functionally discrete subsets (ILC1, ILC2 and ILC3), remarkably similar to helper CD4+ T cell lineages. Many of the transcription factors and cytokines that regulate CD4+ T cell helper subsets also play critical roles in corresponding ILC groups. Thus, T-bet has been shown to be critical for ILC1 development and function, while GATA-3 and RORγt are required for ILC2 and ILC3 function, respectively.

Although ILCs have been grouped into different phenotypic subsets, emerging evidence indicates that ILCs are not ‘fixed’ and that depending on the inflammatory milieu, these cells exhibit considerable functional plasticity. (Artis et al.; Diefenbach, A., Colonna, M., & Koyasu, S. Development, Differentiation, and Diversity of Innate Lymphoid Cells. Immunity 41, 354-365 (2014)). For example, human ILC1s can differentiate into ILC3s in response to local environmental signals, such as IL-1β, retinoic acid, and IL-23. This ability to differentiate into ILC3 is bi-directional, as ILC3s can differentiate into ILC1s in the presence of IL-12. Moreover, gut-resident ILC3s co-express T-bet and RORγt, and have been shown to produce IFNγ in response to microbiota-driven signals, IL-23, or IL-12+ IL-18. Finally, ILC3s can produce IL-5 and IL-13 in response to TLR2 ligands, suggesting that ILC3s may differentiate into ILC2s. However, despite the plasticity that has been demonstrated between ILC1 and ILC3 subsets, it is not clear whether ILC2s exhibit any physiologically relevant functional flexibility. A recent report analyzing gene expression profiles among different ILC subsets revealed that ILC2s were the most homogenous and distinct from the other subsets, (Robinette, M. L. et al. Transcriptional programs define molecular characteristics of innate lymphoid cell classes and subsets. Nat Immunol 16, 306-317 (2015)) consistent with the idea that ILC2s might have a more limited ability to adopt alternative phenotypes compared with ILC1s and ILC3s.

Chronic Obstructive Pulmonary Disease (COPD) is a disorder associated with long-term cigarette smoke exposure, and is characterized by a progressive, irreversible loss of lung function. A subset of COPD patients experience an acute worsening of symptoms following respiratory tract infections, and these exacerbations are a significant cause of morbidity and mortality. The respiratory pathogens influenza, respiratory syncytial virus (RSV), rhinovirus (HRV), and non-typeable Haemophilus influenzae are among the major suspected triggers of COPD-associated exacerbations. COPD is projected to be the third-leading cause of death worldwide by 2030; however, little is understood about how COPD-associated triggers influence immune responses in the lung. A number of IL-1 family member cytokines, including IL-1, IL-18, and IL-33, have been linked to smoke-associated inflammation and exacerbations in mouse models. (Kearley, J. et al. Cigarette Smoke Silences Innate Lymphoid Cell Function and Facilitates an Exacerbated Type I Interleukin-33-Dependent Response to Infection. Immunity 42, 566-579 (2015)). In Kearley et al., it was previously reported that cigarette smoke was associated with a striking redistribution of ST2 receptor expression, the consequence of which altered IL-33-responsiveness in the lung, away from a Th2-associated ILC2 response towards Th1-skewed NK cells and macrophages. Thus, changes in lung-resident ILC populations can have a significant impact on the type and magnitude of the resulting inflammatory response.

The majority of ILCs in the mouse lung are ILC2s, although there are rare but appreciable numbers of type 1 and type 3 ILCs. Cigarette smoke exposure is associated with decreased production of Th2 cytokines by lung-resident ILCs, but the dynamics and developmental relationship between the local ILC subsets in this setting remains unexplored. In the present invention, it has been found that ILC2s exhibit considerable functional plasticity that is dependent on local IL-12 and IL-18 signals that promote the differentiation of ILC2s to ILC1s.

### SUMMARY OF THE INVENTION

ILCs are critical mediators of mucosal immunity, and phenotypic plasticity between group 1 and 3 ILCs has been previously established. Here, it is demonstrated that resident lung ILC2s also exhibit functional plasticity in response to infectious or noxious agents, characterized by the loss of GATA-3 and a concomitant switch to T-Bet⁺ IFN-γ producing ILC1s. The Th1 cytokines, IL-12 and IL-18, induce this conversion, while adoptively transferred GFP+ ILC2s cluster within inflamed areas adopt an ILC1-like phenotype in response to virus. Mechanistically, these ILC1s markedly augment virus-induced inflammation in a T-bet-dependent manner. Notably, IL-12 can convert human ILC2s to T-bet⁺ IFN-γ producing ILC1s, and the frequency of ILC1s in COPD patients correlate with disease severity and susceptibility to exacerbations. Collectively, these data demonstrate that functional plasticity in ILC2s can result in exacerbated anti-viral immunity, which may have adverse consequences in respiratory diseases like COPD.

Some of the main aspects of the present invention are summarized below.

Additional aspects are described in the Detailed Description of the Invention, Examples, Drawings, and Claims sections of this disclosure. The description in each section of this disclosure is intended to be read in conjunction with the other sections. Furthermore, the various embodiments described in each section of this disclosure can be combined in various different ways, and all such combinations are intended to fall within the scope of the present invention.

In one aspect, the disclosure provides a method of inhibiting the conversion of innate lymphoid cells (subset 2) (ILC2s) into innate lymphoid cells (subset 1) (ILC1s) comprising contacting the ILC2s with a modulator that prevents the switch of the ILC2s to T-bet+ IFNγ+ ILC1s, maintains or suppresses the level of IL-12 receptor or IL-18 receptor expression on ILC1s, and/or maintains or increases the level of ST2 or GATA3 expression on ILC2s.

In the disclosed methods, the ILC2s contacted with a modulator can be a local pool of ILC2s, or localized to the lung or lung tissue, or can be circulating ILC2s. In further aspects of the invention the ILC2s inhibited from converting into ILC can be circulating ILC2s, or can be ILC2s that are localized to other tissues, particularly tissues that may be affected or associated with lung inflammatory diseases or COPD.

In other aspects, the disclosure provides a method of preventing or treating a disease or disorder associated with lung inflammation in a subject in need thereof, where the subject is determined to have elevated levels of ILC1s in one or more samples taken from the subject compared to a predetermined level of ILC and/or compared to the levels of ILC in one or more control samples.

In other aspects, the present invention also provides for a method of preventing or treating a disease or disorder associated with lung inflammation in a subject in need thereof, whereby the subject is determined to have an elevated ratio of ILC1s/ILC2s in one or more samples taken from said subject compared to a predetermined ratio of ILC1s/ILC2s and/or compared to the ratio of ILC1s/ILC2s in one or more control samples.

The present invention also provides for a method of preventing or treating exacerbation of a disease or disorder associated with lung inflammation in a subject in need thereof, comprising administering to said subject a disease-modifying medication, whereby said subject is determined to have elevated levels of ILC1s in one or more samples taken from said subject compared to a predetermined level of ILC and/or compared to the levels of ILC in one or more control samples.

In methods of the present invention, lung inflammation can be caused by cigarette smoke, bacterial infection, or viral infection. Further, in methods of the present invention, the disease or disorder is chronic obstructive pulmonary disease (COPD).

The present invention also provides for a method of selecting a patient diagnosed with a disease or disorder associated with lung inflammation as a candidate for treatment with disease-modifying medications, comprising selecting the patient for treatment if the patient is determined to have an elevated ratio of ILC1s/ILC2s in one or more samples taken from said subject compared to a predetermined ratio of ILC1s/ILC2s and/or compared to the ratio of ILC1s/ILC2s in one or more control samples. In certain embodiments, a disease-modifying medication is a modulator of ILC2 to ILC1 conversion, a bronchodilator, an inhaled steroid, a combination inhaler, an oral steroid, or a phosphodiesterase-4 inhibitor.

In particular embodiments, the determination of having an elevated ratio of ILC1s/ILC2s or the determination of having elevated levels of ILC1s is based on a determination of a switch between a molecular signature of ILC2 to a molecular signature of ILC1. In further embodiments, the molecular signature of ILC2 corresponds to the expression of one or more Th2-associated transcripts selected from the group consisting of Gata3, Rora, Il4, Il5, Il9, Il13, Penk (proenkephalin), Areg (amphiregulin), Il17rb, and Il1rI1. In additional embodiments, the molecular signature of ILC1 corresponds to a lower expression of one or more Th2-associated transcripts selected from the group consisting of Gata3, Rora, Il4, Il5, Il9, Il13, Penk (proenkephalin), Areg (amphiregulin), Il17rb, and Il1rI1 and higher levels of one or more transcripts selected from the group consisting of Tbx21, Ifng, Il12rb2, Il18r1, Cxcr3, and Ccr5, wherein said levels are compared to the level of said transcripts in ILC2.

The lung inflammation treated or prevented by the methods of the invention may be caused by cigarette smoke, bacterial infection, viral infection, or a combination thereof. In one embodiment, the disease or disorder treated or prevented by the disclosed methods is chronic obstructive pulmonary disease (COPD).

### BRIEF DESCRIPTION OF THE DRAWINGS

FIG. 1. Influenza infection triggers down-regulation of GATA-3 in lung-resident ILCs. (a) Identification of lung-resident ILCs by flow cytometry as CD45+, Viable, CD3−, CD49b−, Lineage (Lin) negative, CD90⁺. Lineage markers: TCRαβ, TCRγδ, CD5, CD27, F4/80, Gr1, CD19, B220, FCεRI, CD11c and CD11b. (b) Expression of ILC-associated markers on Lin⁻ CD90⁺ lung ILCs (unshaded) compared to isotype controls (gray shaded). (c) Absolute numbers of ILC1, ILC2 and ILC3 in lungs of naïve mice. ILC subsets defined based on expression of T-bet, GATA-3 and RORγt, respectively. (d) Representative FACS of ST2-GFP expression in lung-resident ILCs from ST2-GFP reporter mice. (e) Representative FACS and quantification of intracellular GATA-3 in lung-resident ILCs from naïve mice and mice infected with influenza A. (f) Representative FACS and quantification of T1/ST2 and IL-18Rα expression on lung-resident ILCs in naïve and infected mice. (g) Correlation of ST2 and IL-18Rα expression on lung-resident ILCs in naïve and infected mice. (h) Representative FACS plots and mean fluorescence intensity (MFI) of IL-12432 expression on lung-resident ILCs in naïve and infected mice. (i) Representative FACS plots and quantification of intracellular T-bet expression on lung-resident ILCs in naïve and infected mice at day 7 p.i. (j) Intracellular T-bet expression on IL-18Rα negative or positive ILCs in infected mice at day 7 p.i. (k) Percentage of Ki67+ cells expressing the IL-18Rα in naïve and infected mice. (1) Levels of IL-5, IL-13 and IFN-γ in the supernatants from enriched ILC cultures stimulated as indicated, data presented as change in absolute cytokine levels relative to ILCs from mock-infected mice. Data in a-c and e-i are representative of 5 independent experiments, quantification in d is pooled from 3 independent experiments, j is pooled from two independent experiments and 1-k are representative of two independent experiments. *p<0.01, **p<0.001, *** p<0.0001. Unless otherwise stated, data from infected mice analyzed at Day 7.

FIG. 2. Multiple triggers dramatically alter lung-resident ILC dynamics. (a) GATA-3 MFI of lung-resident ILCs in WT BALB/c mice infected with influenza A (A/FM/1/47), influenza A (PR8) or Respiratory Synctial Virus (RSV), (b) Staphylococcus aureus and (c) Haemophilus influenza. (d-f) Representative FACS and MFI quantification of IL-18Rα and T-bet expression on lung-resident ILCs from naïve and mice infected with H. flu at day 5 post-infection. (g-h) Representative FACS and MFI of GATA-3 expression in lung-resident ILCs from naïve room air controls and mice exposed to cigarette smoke at indicated time points. (i-k) Representative FACS and MFI quantification of T-bet and IL-18Rα expression on lung-resident ILCs from naïve room air controls or mice exposed to cigarette smoke for eight weeks. (1-m) Representative FACS and MFI of IL-12β2 expression on lung-resident ILCs from naïve room air controls or mice exposed to cigarette smoke for eight weeks. Data in a-f are representative of 2-3 independent experiments with n≥5 mice per group. Data in i-m are representative of 3-4 independent experiments with at least 5 mice per group. *p<0.01, **p<0.001, ***p<0.0001.

FIG. 3. (a) Representative flow cytometric plots showing IFN-γ expression from mouse lung ILCs cultured with the indicated cytokines for 96 h. (b) IFN-γ levels in ILC cultures. Representative flow cytometric plots showing expression of GATA-3 (c), or T-bet and IL-18Rα (d) on ILCs from SCID mice intranasally dosed with PBS, IL-33, IL-12+ IL-18 or IL-12+ IL-18+IL-33. (e) Numbers of lung-resident ILC2 (circle) or ILC1 (square) cells in mice treated as indicated. IL-5 (f) and IFN-γ (g) levels in supernatants from ILCs enriched from the lungs of mice in specified treatment groups and stimulated for 24 h as indicated. (h) Representative flow cytometric plot showing cytokine expression on ILCs enriched from the lungs of mice treated with IL-12+ IL-18+IL-33. (i) Representative flow cytometric plots of ST2-GFP versus IL-18Rα expression on lung-resident ILCs from naive littermate controls or ST2-GFP reporter mice or reporter mice treated with IL-12+ IL-18+IL-33. (j) MFI of ST2-GFP expression on ILC2 or ILC1 cells in reporter mice treated as indicated. (k) Cells were sorted from naive lung (ILC2 and NK cells), lungs treated with IL-12+ IL-18+IL-33 (active ILC2 cells (ST2+) and active ILC1 cells (IL-18Rα+)), or naive liver (ILC1 cells) and qPCR analysis of selected genes is visualized as a heat map. Data are expressed as Mean or Mean±S.E.M of 3 (f-g) or 4 (a-d) replicates; or 2 independent experiments (e) with 5 mice/group or 9 mice/group (PBS group only); or pooled from 2 independent experiments (i-j) with 3 (naïve) or 4 (cytokine-treated) mice/group. *p<0.01, **p<0.001.

FIG. 4. Adoptively transferred ILC2s up-regulate ILC1 markers following infection with influenza. (a) Transferred GFP+ ILCs were identifiable via flow cytometry as Lin GFP+ cells expressing CD25, CD90 and ST2. (b) ST2+ IL-18Rα− ILC2s were sorted from the lungs of whole GFP reporter mice treated intranasally with IL-33. 100,000 sorted ILC2s, 30 million splenocytes and 3-4 million C57BL/6 GFP negative lung-derived T/NK cells were transferred intravenously into recipient RAG/SCID deficient mice which were infected with influenza 12 hours later. Flow cytometry of Gata3 (c), IL-18Rα (d) and IL-12Rβ2 (e) on adoptively transferred GFP+ ILCs at Day 7 post-infection. MFI of GATA-3 (f), IL-18Rα (g) and IL-12Rβ2 (h) on adoptively transferred GFP+ ILCs at Day 7 post-infection. Data in b-h are representative of 3 independent experiments with at least 4 mice per group.

FIG. 5. ILC2s cluster in areas associated with viral replication and Th1 cytokine production. (a) Scattergram of individual GFP+ cells and their mean expression of GATA-3 and nuclear content, measured as mean intensity of the DNA probe DAPI (dark grey) in automatically segmented GFP+ cell masks (ROIs; see methods). (b) Histogram showing the frequency of pooled GFP+ cells (y axis) in control and infected mice against GATA-3 values adjusted for nuclear DAPI content. (c) Quantification of ILC GATA-3 expression in individual mice at 6d post infection. Data in a-c represent 700-1700 total counted cells per condition.

FIG. 6. ILC1 cells augment anti-viral immunity in a T-bet-dependent manner. Quantification and representative flow cytometric plots of (a-b) GATA-3, (c-d) IL-12Rβ2, and (e-f) ST2 and IL-18Rα expression in lung-resident ILCs in C57BL/6 and Tbx21−/− mice at day 7 post-challenge with influenza PR8. (g) Representative flow cytometric plots showing IFN-γ expression in ILCs from naive and infected C57BL/6 and Tbx21−/− mice enriched at day 7 post-infection. ILC2 and ILC1 cells were purified from mice treated with IL-33 or IL-33+IL-12+ IL-18, respectively, and (h) transferred into RAG/γ c-deficient mice. Virus-induced weight loss and (i) BAL cytokine expression measured at day 2 post-infection (j) in RAG/γ c-deficient mice reconstituted with ILC2 or ILC1 cells purified from cytokine-treated mice (see methods). (k) Expression of cytokines in the BAL fluid of RAG/γ c-deficient mice reconstituted with ILC1 cells purified from C57BL/6 or Tbx21−/− mice measured at day 2 post-infection. Data in a-g are representative of two independent experiments with 5 (C57BL/6), 6 (infected Tbx21−/−) or 7 (naive Tbx21−/−) mice/group. Data in i-j are representative of two independent experiments with 4 (ILC2 reconstituted, ILC1 reconstituted control) or 5 (ILC1 reconstituted infected) mice/group. Data in k is from one experiment with 7 mice/group. *p<0.01, **p<0.001.

FIG. 7. IL-12 suppresses GATA-3 and induces T-bet and IFN-γ in human ILC2s. ILC2s, defined as CD45+ viable CD3⁻ CD19⁻ Lin⁻ IL-7Rα⁺ CD161⁺ CRTH2⁺, were sorted from the peripheral blood of normal human donors and cultured for 5 days with IL-33, IL-12 or IL-33+IL-12. All cultures contained IL-2. (a) GATA-3, (b) T-bet and (c) CD25 expression on ILC2s after 5 days of culture in indicated conditions. Levels of (d) IL-13, (e) IL-4 and (f) IFN-γ were measured in culture supernatants by ELISA. Data are representative of 4 independent experiments and cells were pooled from 3-4 healthy donors for each experiment.

FIG. 8. COPD patients have augmented percentages of circulating ILC1 which correlates with disease severity. (a) Representative T-bet and CRTH2 expression on ILCs in peripheral blood of a non-smoking control and COPD patient. (b) Percentage of circulating ILC1 in healthy controls, smoking controls and total COPD patients; ILC1 defined as T-bet⁺. (c) Percentage of circulating ILC2 in healthy controls, smoking controls and total COPD patients; ILC2 defined as CRTH2⁺. (d) Percentage of circulating ILC1 and (e) ILC2 among healthy controls, smoking controls and COPD patients stratified by GOLD status. (f) Correlation between percentage of ILC1 and FEV1% Predicted. (g) Percentage of circulating ILC1 and (h) ILC2 among healthy controls, smoking controls and COPD patients stratified by exacerbation frequency. (i) Correlation between percentages of circulating ILC2 and ILC1 in healthy controls, smoking controls and total COPD patients. Abbreviations in the figure: NS=non-smokers; Sm=smokers. *p<0.05, **p<0.01, ***p<0.001.

### DETAILED DESCRIPTION OF THE INVENTION

The present invention demonstrates that influenza-associated inflammation causes significant phenotypic changes within the lung ILC population, characterized by a loss of GATA-3 and a decrease in the expression of Th2-associated markers, which strongly correlates with a marked expansion of IL-18Rα+T-bet⁺ ILC1s. Further, also provided is novel evidence, utilizing an ST2-GFP reporter mouse and adoptive transfer of GFP+ ILC2s, that ILC1 expansion is a result of direct conversion from resident lung ILC2s which occurs in response to IL-12 and IL-18 stimulation. It is further shown that a wide variety of triggers, especially those associated with exacerbations of COPD, initiate this plasticity in local ILC2s, including a number of viral and bacterial infections as well as cigarette smoke exposure. Notably, the magnitude of insults that trigger this functional conversion of ILC2s suggests that this response is a general feature of inflammation and/or damage in the lung.

Moreover, the present invention provides that ILC2 plasticity involves a multi-step sequence of events: migration of ILC2s to inflamed areas within the tissue, silencing via the significant loss of GATA-3, and subsequent exposure to microenvironmental cues that dictate the phenotypic switch and functional local response of these cells. Using an adoptive transfer system with GFP+ ILC2s, the present invention provides that following infection, ‘silenced’ ILCs (i.e., GATA-3^{LOW}) converge within the inflammatory areas associated with local IL-12 and IL-18 production, and switch to an ILC1 phenotype. Molecular analyses following IL-12+ IL-18+IL-33 stimulation aligns with this concept for ILC2 plasticity, since the emerging ILC1s (i.e., ex-ILC2s) share a partially overlapping, but distinct, gene signature with activated ILC2s. This is characterized by a dampening of Th2 transcripts and a pronounced increase in Th1-like genes associated with a pro-inflammatory response. Importantly, IL-12+ IL-18+IL-33 activated ILC2s exhibit a mixed Th1/Th2 signature, including the co-expression of the transcription factors GATA-3 and T-bet, compared to resting ILC2s, indicating that lung ILC2 plasticity occurs in response to local microenvironmental changes.

Interestingly, upregulation of the receptors for IL-12 and IL-18 occurs on ILCs during bacterial infection, as well as cigarette smoke exposure, indicating that these cytokines are associated with ILC1 conversion in multiple settings. In addition, these Th1-like inflammatory cytokines have been shown to be essential in the host response to bacterial infection and smoke induced inflammation. Thus, the ability of ILC2s to switch phenotypes appears to require multiple signals and involve triggers associated with pulmonary diseases, such as COPD.

The striking similarity of the response from lung-resident ILC populations to infectious and noxious agents supports a common mechanism underlying these phenotypic changes. IL-12 has recently emerged as a key regulator of ILC3 plasticity in humans, and data indicates a role for this cytokine in directly inducing plasticity of mouse and human ILC2s. Indeed, IL-12 is produced during the viral and bacterial infections used here, and intranasal administration of IL-12 and IL-18 results in a very similar phenotype, i.e., loss of GATA-3 and specific emergence of ILC1s. Additionally, local administration of exogenous IL-12 during viral challenge enhances the loss of GATA-3 and augments the subsequent ILC1 expansion. The early expression of IL-12Rβ2 on ILC2s before IL-18Rα, and the presence of ILC2s co-expressing ST2 and IL-12Rβ2 are consistent with the observation that IL-12 can contribute to the initial silencing and phenotypic switch in these cells. One of the key downstream effectors induced by IL-12 is IFN-γ, and recent reports indicate that this cytokine can directly dampen ILC2 responses. (Molofsky, A. et al. Interleukin-33 and Interferon-γ Counter-Regulate Group 2 Innate Lymphoid Cell Activation during Immune Perturbation. Immunity 43, 161-174 (2015)). Thus, the IL-12-IFN-γ axis appears to be a critical factor in the regulation of ILC2 silencing and phenotypic switch to ILC1.

Infection with most viruses, including influenza, causes lung disease characterized by histologically distinct loci of inflammation associated with areas of viral replication. Despite low numbers of ILCs, extensive immunohistochemical image analysis performed in the present studies, revealed that after infection, ILCs converge within the inflamed areas of the tissue. The present invention discloses a first instance of specifically visualizing ILCs in the lung and, importantly, the data revealed that these cells cluster within influenza-dense areas in close vicinity to myeloid-derived cells expressing IL-12 and IL-18 mRNA. This accumulation of ILCs, in addition to the high cytokine output of these cells, implies that the microenvironmental cytokine concentrations would be markedly enhanced during infection. Indeed, adoptively transferred ILC1s significantly amplified the Th1-like inflammatory cytokine production, including TNFα, IL-1β, IL-12p70 and IFN-γ, evoked by virus infection, and the viral-induced weight loss when compared to animals that received ILC2s prior to infection.

Remarkably, it was also found that ILCs associated with the infected regions of the lung had lower expression of GATA-3 than those found in the unaffected areas of the tissue. Taken together, these data demonstrate that ILC2s encountering inflammatory loci are actively silenced and locally directed to switch phenotype and produce alternative mediators thus amplifying viral induced immunity. The extensive IHC image analyses of the present invention have highlighted the importance of examining local changes, since whole population-level analysis, i.e., total lung digests, may obscure or under-estimate micro-environmental changes in tissue-resident ILC phenotypes.

Long term cigarette smoke exposure can lead to the development of COPD, and has been linked to the subsequent susceptibility to exacerbation in a subset of these patients. Surprisingly, relatively little is understood about how the pulmonary immune system responds to this noxious agent. An important finding provided herein was that smoke exposure also triggers the conversion of resident lung ILC2s to ILC1s, and thereby potentially increasing susceptibility to injury and aggravated inflammation observed in chronic smokers. Mechanistically, these data also provide underlying context for the silencing effect observed on ILC2s isolated from the lungs of smoke-exposed mice, where the production of IL-5 and IL-13 are markedly reduced after ex vivo stimulation. IL-12 appears to be a critical regulator of ILC2 plasticity in infectious settings but this cytokine has also been shown to be produced in response to cigarette smoke exposure. Further, IL-18 and IL-33, two factors that augment IL-12-induced ILC2 plasticity, are both markedly up-regulated during experimental smoke exposure and in COPD patients. Thus, an IL-12-inducing respiratory infection may be primed to drive hyper-inflammatory ILC1 responses in the context of elevated IL-33 and IL-18 associated with prior cigarette smoke exposure.

Previous studies have shown that culture of human ILC3s with IL-12 results in the up-regulation of ILC1 markers, while ILC can differentiate into ILC3s in the presence of IL-23, IL-1 (3, and retinoic acid. The data in the present invention demonstrate that human CRTH2+ ILC2s respond to IL-12 by down-regulating GATA-3 and up-regulating T-bet and IFN-γ. These cells also lose CD25, which is consistent with the lower expression of CD25 in IL-18Rα⁺ mouse ILC1s. Furthermore, previous studies confirm that human ILC2s can switch to ILC1-IFN-γ producing cells following IL-12 and/or IL-1β+IL-12 stimulation. It is now apparent that all human ILC subsets exhibit functional plasticity, which raises the question of how frequently ILCs flip phenotypes in disease states.

Here, findings have also been extended to human COPD, showing that patients have significantly altered ratios of circulating ILC2s and ILC1s. Specifically, patients with higher numbers of circulating ILC have worse lung function, more severe disease, and appear to be more susceptible to frequent exacerbations. Hence, the circulating ILC subtypes may be reflective of the local ILC populations in this disease. Given the ability of ILC1s to dramatically amplify infection-induced inflammation in the mouse, it is possible that increased numbers of ILC1s in COPD patients may be causative in the frequency or severity of exacerbations. The strong correlation between ILC1s and ILC2s in COPD patients, echoing the relationship between ST2+ and IL-18Rα⁺ ILCs in inflamed mouse lung, indicates that plasticity could be an active process in this disease. Given the phenotypically promiscuous nature of ILCs, and the lack of known cell-specific surface markers for depletion, targeting these cells through manipulation of their plasticity, i.e., by converting hyper-inflammatory ILC1s back to tissue-protective ILC2s, may offer novel therapeutic approaches in the treatment and management of exacerbations in COPD.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, e.g., Ausubel et al. eds. (2015) Current Protocols in Molecular Biology (John Wiley and Sons); Greenfield, ed. (2013) Antibodies: A Laboratory Manual (2nd ed., Cold Spring Harbor Press); Green and Sambrook, eds. (2012), Molecular Cloning: A Laboratory Manual (4th ed., Cold Spring Harbor Laboratory Press); Krebs et al., eds. (2012) Lewin's Genes XI (11th ed., Jones & Bartlett Learning); Freshney (2010) Culture Of Animal Cells (6th ed., Wiley); Weir and Blackwell, eds., (1996) Handbook Of Experimental Immunology, Volumes I-IV (5th ed., Wiley-Blackwell); Borrebaeck, ed. (1995) Antibody Engineering (2nd ed., Oxford Univ. Press); Glover and Hames, eds., (1995) DNA Cloning: A Practical Approach, Volumes I and II (2nd ed., IRL Press); Rees et al., eds. (1993) Protein Engineering: A Practical Approach (1st ed., IRL Press); Mayer and Walker, eds. (1987) Immunochemical Methods In Cell And Molecular Biology (Academic Press, London); Nisonoff (1984) Introduction to Molecular Immunology (2nd ed., Sinauer Associates, Inc.); and Steward (1984) Antibodies: Their Structure and Function (1st ed., Springer Netherlands).

In order that the present invention can be more readily understood, certain terms are defined. Additional definitions are set forth throughout the disclosure. Unless defined otherwise, all technical and scientific terms used herein have the same meaning as commonly understood by one of ordinary skill in the art to which this invention is related. For example, The Dictionary of Cell and Molecular Biology (5th ed. J. M. Lackie ed., 2013), the Oxford Dictionary of Biochemistry and Molecular Biology (2d ed. R. Cammack et al. eds., 2008), and The Concise Dictionary of Biomedicine and Molecular Biology (2d ed. P-S. Juo, 2002) can provide one of skill with general definitions of some terms used herein.

### Definitions

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Units, prefixes, and symbols are denoted in their Systeme International de Unites (SI) accepted form. Numeric ranges are inclusive of the numbers defining the range. The headings provided herein are not limitations of the various aspects or embodiments of the invention, which can be had by reference to the specification as a whole. Accordingly, the terms defined immediately below are more fully defined by reference to the specification in its entirety.

Wherever embodiments are described with the language “comprising,” otherwise analogous embodiments described in terms of “consisting of” and/or “consisting essentially of” are included.

Amino acids are referred to herein by their commonly known three-letter symbols or by the one-letter symbols recommended by the IUPAC-IUB Biochemical Nomenclature Commission. Nucleotides, likewise, are referred to by their commonly accepted single-letter codes.

“IL-12” refers to interleukin 12. The active form of IL-12 is a heterodimer; the p35 subunit is encoded by the IL-12A gene, and the p40 subunit is encoded by the IL-12B gene. The full-length amino acid and nucleotide sequences for both subunits of human and other mammalian IL-12 are known in the art. IL-12 binds to a type I cytokine receptor. The IL-12 receptor (IL-12R) is a transmembrane protein comprised of β1 and β2 subunits. The full-length amino acid and nucleotide sequences for human and other mammalian IL-12(31 and IL-12(32 are known in the art.

“IL-18” refers to interleukin 18, also called IFN-γ inducing factor. The full-length amino acid and nucleotide sequences of human and other mammalian IL-18 are known in the art. IL-18 binds to the IL-18 receptor (IL-18R), which is a heteromeric complex comprised of the IL-18 receptor accessory protein (IL-18RAP) and the IL-18 receptor 1 (IL-18R1) protein. The full-length amino acid and nucleotide sequences for human and other mammalian IL-18RAP and IL-18R1 are known in the art.

An “inhibitor” is a molecule that inhibits, blocks, or suppresses the activity of another molecule. The activity of ligands can be inhibited, for instance, by interfering with the binding of the ligand to its receptor, or by interfering with binding-induced activation of the receptor. Inhibition can be achieved by blocking the ligand itself, or by blocking the receptor to which it binds. Moreover, in the case of transmembrane receptors, the introduction of soluble receptor derivatives can inhibit the ligand's activity. Soluble receptor derivatives compete for ligand binding with native transmembrane receptors, thus reducing or eliminating cell activation or signaling resulting from the ligand binding to the native receptors. Inhibition can be agonistic or antagonistic. An inhibitor can be, for example, a small molecule, a binding molecule, including muteins and antibodies or antigen-binding fragment thereof, an inhibitory RNA, or an antisense oligonucleotide. Inhibitors for use in the invention are preferably “specific,” meaning that they exert an effect on one target or on a group of structurally related targets.

The terms “inhibit,” “block,” and “suppress” are used interchangeably and refer to any statistically significant decrease in biological activity, including full blocking of the activity. For example, “inhibition” can refer to a decrease of about 10%, 20%, 30%, 40%, 50%, 60%, 70%, 80%, 90% or 100% in biological activity. Accordingly, when the terms “inhibition” or “suppression” are applied to describe, e.g., an effect on a signal transduction pathway, the terms refer to the ability of an inhibitor to statistically significantly decrease a signal- or target-induced cell development, plasticity, or signal transduction relative to an untreated (control) cell. In certain embodiments, an inhibitor can inhibit target-mediated cell activation or signal transduction in target-responsive cell by at least 10%, or at least 20%, or at least 30%, or at least 40%, or at least 50%, or at least 60%, or at least 70%, or at least 80%, or at least 90% or about 100%, as determined, for example, by flow cytometry, Western blotting, ELISA, or other assays known to those of skill in the art. A “blocking” antibody or an “antagonist” antibody is one that inhibits or reduces biological activity of the antigen it binds. In certain aspects, blocking antibodies or antagonist antibodies substantially or completely inhibit the biological activity of the antigen. Desirably, the biological activity is reduced by 10%, 20%, 30%, 40%, 50%, 60%, 70%, 80%, 90%, 95%, or even 100%.

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Low-affinity antibodies generally bind antigen slowly and tend to dissociate readily, whereas high-affinity antibodies generally bind antigen faster and tend to remain bound longer.

The affinity or avidity of an antibody for an antigen can be determined experimentally using any suitable method known in the art, e.g., flow cytometry, enzyme-linked immunosorbent assay (ELISA), or radioimmunoassay (RIA), or kinetics (e.g., KINEXA® or BIACORE™ analysis). Direct binding assays as well as competitive binding assay formats can be readily employed. (See, e.g., Berzofsky et al., “Antibody-Antigen Interactions,” In Fundamental Immunology, Paul, W. E., ed., Raven Press: New York, N.Y. (1984); Kuby, Immunology, W. H. Freeman and Company: New York, N.Y. (1992)) The measured affinity of a particular binding molecule-target interaction can vary if measured under different conditions (e.g., salt concentration, pH, temperature). Thus, measurements of affinity and other target-binding parameters (e.g., K_{D} or Kd, Kₒₙ, K_{off}) are made with standardized solutions of binding molecule (e.g., antibody) and target (e.g., antigen), and a standardized buffer, as known in the art.

The term “modulator” refers to a molecule or compound that causes a change (e.g, an inhibition, suppression, stimulation or increase in activity) to a metabolic pathway. A “modulator” can be, for example, a small molecule, a polypeptide, an antibody, an antigen binding fragment, or mutein.

A “small molecule” is typically an organic molecule of low molecular weight, i.e., less than about a kilodalton. Small molecule inhibitors can be identified, for example, by screening small molecule libraries using routine methods.

A “binding molecule” is one that is capable of binding its target with sufficient affinity such that the binding molecule is useful as a therapeutic agent or diagnostic reagent. A binding molecule that “specifically binds” to its target binds to an unrelated protein to an extent of less than about 10% of the binding of the binding molecule to its target, as measured, e.g., by a radioimmunoassay (RIA), BIACORE®, KINEXA®, or other binding assays known in the art. In certain embodiments, the binding molecule binds to its target with a dissociation constant (K_{D}) of ≤1 μM, —≤100 nM, ≤10 nM, ≤1 nM, ≤0.1 nM, ≤10 μM, ≤1 μM, or ≤0.1 μM. The term “binding molecule” includes antibodies and antigen-binding fragments thereof. In certain embodiments, the binding molecule is a polypeptide that is not an antibody. A variety of methods for identifying and producing non-antibody polypeptides that bind with high affinity to a protein target are known in the art. See, e.g., Skerra, Curr. Opin. Biotechnol. 18:295-304 (2007), Hosse et al., Protein Science 15:14-27 (2006), Gill et al., Curr. Opin. Biotechnol. 17:653-658 (2006), Nygren, FEBS J. 275:2668-76 (2008), and Skerra, FEBS J. 275:2677-83 (2008). In certain embodiments, phage display technology can been used to identify and/or produce a binding molecule, such as a polypeptide. In certain embodiments, the polypeptide comprises a protein scaffold of a type selected from the group consisting of protein A, a lipocalin, a fibronectin domain, an ankyrin consensus repeat domain, and thioredoxin.

A “mutein” is an analog of a naturally occurring protein, in which one or more amino acid residues are added to, deleted from, or replaced by different amino acid residues, relative to the natural amino acid sequence. Muteins can be prepared by known synthetic methods, for example, as described herein, by site-directed mutagenesis, or by any other suitable technique known in the art.

The term “antibody” refers to an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses polyclonal antibodies; monoclonal antibodies; multispecific antibodies, such as bispecific antibodies generated from at least two intact antibodies; humanized antibodies; human antibodies; chimeric antibodies; fusion proteins comprising an antigen-determination portion of an antibody; and any other modified immunoglobulin molecule comprising an antigen recognition site, so long as the antibodies exhibit the desired biological activity. Antibodies can be of any the five major classes of immunoglobulins: IgA, IgD, IgE, IgG and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu respectively. The different classes of immunoglobulins have different and well-known subunit structures and three-dimensional configurations. There are two classes of mammalian light chains, lambda and kappa. Antibodies can be naked or conjugated to other molecules such as toxins, radioisotopes, etc.

The term “antigen-binding fragment” refers to a portion of an intact antibody comprising the complementarity determining variable regions of the antibody. Fragments of a full-length antibody can be an antigen-binding fragment of an antibody. Examples of antibody fragments include, but are not limited to Fab, Fab′, F(ab′)2, and Fv fragments, linear antibodies, single chain antibodies (e.g., ScFvs), and multispecific antibodies formed from antibody fragments.

A “monoclonal antibody” (mAb) refers to a homogeneous antibody population that is involved in the highly specific recognition and binding of a single antigenic determinant, or epitope. This is in contrast to polyclonal antibodies, which typically include different antibodies directed against different antigenic determinants. The term “monoclonal” can apply to both intact and full-length monoclonal antibodies, as well as to antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal antibody” refers to such antibodies made in any number of ways including, but not limited to, by hybridoma, phage selection, recombinant expression, and transgenic animals.

The term “humanized antibody” refers to an antibody derived from a non-human (e.g., murine) immunoglobulin, which has been engineered to contain minimal non-human (e.g., murine) sequences. Typically, humanized antibodies are human immunoglobulins in which residues from the complementary determining region (CDR) are replaced by residues from the CDR of a non-human species (e.g., mouse, rat, rabbit, or hamster) that have the desired specificity, affinity, and capability (Jones et al., 1986, Nature, 321:522-525; Riechmann et al., 1988, Nature, 332:323-327; Verhoeyen et al., 1988, Science, 239:1534-1536). In some instances, the Fv framework region (FW) residues of a human immunoglobulin are replaced with the corresponding residues in an antibody from a non-human species that has the desired specificity, affinity, and capability.

Humanized antibodies can be further modified by the substitution of additional residues either in the Fv framework region and/or within the replaced non-human residues to refine and optimize antibody specificity, affinity, and/or capability. In general, humanized antibodies will comprise substantially all of at least one, and typically two or three, variable domains containing all or substantially all of the CDR regions that correspond to the non-human immunoglobulin whereas all or substantially all of the FR regions are those of a human immunoglobulin consensus sequence. Humanized antibody can also comprise at least a portion of an immunoglobulin constant region or domain (Fc), typically that of a human immunoglobulin. Examples of methods used to generate humanized antibodies are described in U.S. Pat. Nos. 5,225,539 and 5,639,641.

The term “human antibody” means an antibody produced by a human or an antibody having an amino acid sequence corresponding to an antibody produced by a human made using any technique known in the art. The definition of a human antibody includes intact or full-length antibodies comprising at least one human heavy and/or light chain polypeptide such as, for example, an antibody comprising murine light chain and human heavy chain polypeptides.

The term “chimeric antibodies” refers to antibodies wherein the amino acid sequence of the immunoglobulin molecule is derived from two or more species. Typically, the variable region of both light and heavy chains corresponds to the variable region of antibodies derived from one species of mammals (e.g., mouse, rat, rabbit, etc.) with the desired specificity, affinity, and capability while the constant regions are homologous to the sequences in antibodies derived from another (usually human) to avoid eliciting an immune response in that species.

The terms “antibody” or “immunoglobulin” are used interchangeably herein. A typical antibody comprises at least two heavy (H) chains and two light (L) chains interconnected by disulfide bonds. Each heavy chain is comprised of a heavy chain variable region (abbreviated herein as VH) and a heavy chain constant region. The heavy chain constant region is comprised of three domains, CH1, CH2, and CH3. Each light chain is comprised of a light chain variable region (abbreviated herein as VL) and a light chain constant region (CL). The light chain constant region is comprised of one domain, Cl. The variable regions of the heavy and light chains contain a binding domain that interacts with an antigen. The constant regions of the antibodies can mediate the binding of the immunoglobulin to host tissues or factors, including various cells of the immune system (e.g. effector cells) and the first component (C1q) of the classical complement system.

The VH and VL regions can be further subdivided into regions of hypervariability, termed complementarity-determining regions (CDRs), interspersed with regions that are more conserved, termed framework (FW) regions. The CDRs in each chain are held together in close proximity by the FW regions and, with the CDRs from the other chain, contribute to the formation of the antigen-binding site of antibodies. Each VH and VL is composed of three CDRs and four FWs, arranged from amino-terminus to carboxy-terminus in the following order: FW1, CDR1, FW2, CDR2, FW3, CDR3, FW4.

There are at least two techniques for determining CDRs: (1) an approach based on cross-species sequence variability (Kabat et al., Sequences of Proteins of Immunological Interest, 5th Ed. Public Health Service, National Institutes of Health, Bethesda, Md. (1991)); and (2) an approach based on crystallographic studies of antigen-antibody complexes (Al-lazikani et al., J. Molec. Biol. 273:927-948 (1997)). In addition, combinations of these two approaches are sometimes used in the art to determine CDRs.

The amino acid position numbering as in Kabat, refers to the numbering system used for heavy chain variable domains or light chain variable domains (approximately residues 1-107 of the light chain and residues 1-113 of the heavy chain). Using this numbering system, the actual linear amino acid sequence can contain fewer or additional amino acids corresponding to a shortening of, or insertion into, a FW or CDR of the variable domain. The Kabat numbering of residues can be determined for a given antibody by alignment at regions of homology of the sequence of the antibody with a “standard” Kabat numbered sequence.

An “inhibitory RNA” is one which inhibits gene expression by RNA interference.

Examples of inhibitory RNAs include micro RNA (miRNA) and small interfering RNA (siRNA). An “antisense oligonucleotide” is a nucleic acid strand that binds to mRNA and prevents its translation. Antisense oligonucleotides can be comprised of ribonucleotides, deoxyribonucleotides, nucleotide analogs, or combinations thereof. Methods for designing and producing inhibitory RNA and antisense oligonucleotides are known in the art.

An “isolated” polypeptide, antibody, binding molecule, polynucleotide, vector, or cell is in a form not found in nature. Isolated polypeptides, antibodies, binding molecules, polynucleotides, vectors, or cells include those which have been purified to a degree that they are no longer in a form in which they are found in nature. In some embodiments, a polypeptide, antibody, binding molecule, polynucleotide, vector, or cell that is isolated is substantially pure. When used herein. the term “substantially pure” refers to purity of greater than 75%, preferably greater than 80% or 90%, and most preferably greater than 95%.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, sports animals, and zoo animals including, e.g., humans, non-human primates, dogs, cats, guinea pigs, rabbits, rats, mice, horses, cattle, bears, and so on.

The term “pharmaceutical composition” refers to a preparation that is in such form as to permit the biological activity of the active ingredient to be effective and which contains no additional components that are unacceptably toxic to a subject to which the composition would be administered. Such composition can be sterile and can comprise a pharmaceutically acceptable carrier, such as physiological saline. Suitable pharmaceutical compositions can comprise one or more of a buffer (e.g. acetate, phosphate or citrate buffer), a surfactant (e.g. polysorbate), a stabilizing agent (e.g. human albumin), a preservative (e.g. benzyl alcohol), an absorption promoter to enhance bioavailability and/or other conventional solubilizing or dispersing agents.

An “effective amount” of a binding molecule as disclosed herein is an amount sufficient to carry out a specifically stated purpose, e.g., a therapeutic or prophylactic effect. An “effective amount” can be determined empirically and in a routine manner, in relation to the stated purpose.

Terms such as “treating” or “treatment” or “to treat” or “alleviating” or “to alleviate” refer to therapeutic measures that cure, slow down, lessen symptoms of, and/or halt progression of a diagnosed pathologic condition or disorder. Thus, those in need of treatment include those already with the disorder. In certain embodiments, a subject is successfully “treated” for a disease or disorder associated with lung inflammation according to the methods provided herein if the patient shows, e.g., total, partial, or transient alleviation or elimination of symptoms associated with the disease or disorder.

“Prevent” or “prevention” refer to prophylactic or preventative measures that prevent and/or slow the development of a targeted pathologic condition or disorder. Thus, those in need of prevention include those prone to have or susceptible to the disorder. In certain embodiments, a disease or disorder associated with lung inflammation is successfully prevented according to the methods provided herein if the patient develops, transiently or permanently, e.g., fewer or less severe symptoms associated with the disease or disorder, or a later onset of symptoms associated with the disease or disorder, than a patient who has not been subject to the methods of the invention.

The terms “polypeptide,” “peptide,” and “protein” are used interchangeably herein to refer to polymers of amino acids of any length. The polymer can be linear or branched, it can comprise modified amino acids and non-amino acids can interrupt it. The terms also encompass an amino acid polymer that has been modified naturally or by intervention; for example, disulfide bond formation, glycosylation, lipidation, acetylation, phosphorylation or any other manipulation or modification such as conjugation with a labeling component. Also included within the definition are, for example, polypeptides containing one or more analogs of an amino acid (including, for example, unnatural amino acids, etc.), as well as other modifications known in the art. In certain embodiments, the polypeptides can occur as single chains or associated chains.

A “polynucleotide,” as used herein can include one or more “nucleic acids,” “nucleic acid molecules,” or “nucleic acid sequences,” and refers to a polymer of nucleotides of any length, and includes DNA and RNA. The polynucleotides can be deoxyribonucleotides, ribonucleotides, modified nucleotides or bases, and/or their analogs, or any substrate that can be incorporated into a polymer by DNA or RNA polymerase. A polynucleotide can comprise modified nucleotides, such as methylated nucleotides and their analogs. The preceding description applies to all polynucleotides referred to herein, including RNA and DNA.

### Preparation of Binding Molecules

Monoclonal antibodies can be prepared using hybridoma methods, such as those described by Kohler and Milstein Nature 256:495 (1975). Using the hybridoma method, a mouse, hamster, or other appropriate host animal, is immunized to elicit the production by lymphocytes of antibodies that will specifically bind to an immunizing antigen. Lymphocytes can also be immunized in vitro. Following immunization, the lymphocytes are isolated and fused with a suitable myeloma cell line using, for example, polyethylene glycol (PEG), to form hybridoma cells that can then be selected away from unfused lymphocytes and myeloma cells. Hybridomas that produce monoclonal antibodies directed specifically against a chosen antigen as determined by immunoprecipitation, immunoblotting, or by an in vitro binding assay (e.g. RIA or ELISA) can then be propagated either in in vitro culture using standard methods (Goding, Monoclonal Antibodies: Principles and Practice, Academic Press, 1986) or in vivo as ascites tumors in an animal. The monoclonal antibodies can then be purified from the culture medium or ascites fluid.

Human antibodies can be directly prepared using various techniques known in the art. Immortalized human B lymphocytes immunized in vitro or isolated from an immunized individual that produce an antibody directed against a target antigen can be generated (See, e.g., Cole et al., Monoclonal Antibodies and Cancer Therapy, Alan R. Liss, p. 77 (1985); Boemer et al., J. Immunol. 147 (1):86-95 (1991); and U.S. Pat. No. 5,750,373).

The inhibitor can be selected from a phage library, where the phage library expresses human antibodies, as described, for example, by Vaughan et al. (Nat. Biotechnol., 14:309-314 (1996)), Sheets et al. (Proc. Nat'l. Acad. Sci. U.S.A. 95:6157-6162 (1998)), Hoogenboom et al. (J. Mol. Biol. 227:381 (1991)), and Marks et al. (J. Mol. Biol. 222:581 (1991)). Techniques for the generation and use of antibody phage libraries are also described in U.S. Pat. Nos. 5,969,108, 6,172,197, 5,885,793, 6,521,404; 6,544,731; 6,555,313; 6,582,915; 6,593,081; 6,300,064; 6,653,068; 6,706,484; and 7,264,963; and in Rothe et al., J. Mol. Biol. 375:1182-1200 (2007).

Affinity maturation strategies and chain shuffling strategies are known in the art and can be employed to generate high affinity human antibodies or antigen-binding fragments thereof. (See Marks et al., Bio/Technology 10:779-783 (1992)).

In some embodiments, the antibody can be a humanized antibody or antigen-binding fragment thereof. Methods for engineering, humanizing, or resurfacing non-human or human antibodies can also be used and are well known in the art. A humanized, resurfaced, or similarly engineered antibody can have one or more amino acid residues from a source that is non-human, e.g., mouse, rat, rabbit, non-human primate, or other mammal. These non-human amino acid residues are replaced by residues that are often referred to as “import” residues, which are typically taken from an “import” variable, constant, or other domain of a known human sequence. Such imported sequences can be used to reduce immunogenicity or reduce, enhance, or modify binding, affinity, on-rate, off-rate, avidity, specificity, half-life, or any other suitable characteristic, as known in the art. In general, the CDR residues are directly and most substantially involved in influencing antigen binding. Accordingly, part or all of the non-human or human CDR sequences are maintained while the non-human sequences of the variable and constant regions can be replaced with human or other amino acids.

Antibodies can also optionally be humanized, resurfaced, engineered, or human antibodies engineered with retention of high affinity for the target antigen and other favorable biological properties. To achieve this goal, humanized (or human) or engineered antibodies and resurfaced antibodies can be optionally prepared by a process of analyzing the parental sequences and various conceptual humanized and engineered products, using three-dimensional models of the parental, engineered, and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate binding molecule sequence, i.e., the analysis of residues that influence the ability of the candidate binding molecule to bind its target. In this way, FW residues can be selected and combined from the consensus and import sequences so that the desired binding molecule characteristic, such as increased affinity for the target, is achieved.

Humanization, resurfacing, or engineering of antibodies or antigen-binding fragments thereof can be performed using any known method, such as, but not limited to, those described in, Jones et al., Nature 321:522 (1986); Riechmann et al., Nature 332:323 (1988); Verhoeyen et al., Science 239:1534 (1988)), Sims et al., J. Immunol. 151: 2296 (1993); Chothia and Lesk, J. Mol. Biol. 196:901 (1987), Carter et al., Proc. Natl. Acad. Sci. U.S.A. 89:4285 (1992); Presta et al., J. Immunol. 151:2623 (1993), U.S. Pat. Nos. 5,639,641, 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; 4,816,567, 7,557,189; 7,538,195; and 7,342,110; International Application Nos. PCT/US98/16280; PCT/US96/18978; PCT/US91/09630; PCT/US91/05939; PCT/US94/01234; PCT/GB89/01334; PCT/GB91/01134; PCT/GB92/01755; International Patent Application Publication Nos. WO90/14443; WO90/14424; WO90/14430; and European Patent Publication No. EP 229246.

Humanized antibodies and antigen-binding fragments thereof can also be made in transgenic mice containing human immunoglobulin loci that are capable, upon immunization, of producing the full repertoire of human antibodies in the absence of endogenous immunoglobulin production. This approach is described in U.S. Pat. Nos. 5,545,807; 5,545,806; 5,569,825; 5,625,126; 5,633,425; and 5,661,016.

In certain embodiments an antibody fragment is used as. Various techniques are known for the production of antibody fragments. Traditionally, these fragments are derived via proteolytic digestion of intact antibodies. See, e.g., Morimoto et al., J. Biochem. Biophys. Meth. 24:107-117 (1993); Brennan et al., Science, 229:81-83 (1985). In certain embodiments, antibody fragments are produced recombinantly. Fab, Fv, and scFv antibody fragments can all be expressed in and secreted from E. coli or other host cells, thus allowing the production of large amounts of these fragments. Such antibody fragments can also be isolated from the antibody phage libraries discussed above. Antibody fragments can also be linear antibodies, as described in U.S. Pat. No. 5,641,870. Other techniques for the production of antibody fragments will be apparent to the skilled practitioner.

According to the present invention, techniques can be adapted for the production of single-chain antibodies specific to a given target (see, e.g., U.S. Pat. No. 4,946,778). In addition, methods can be adapted for the construction of Fab expression libraries (see, e.g., Huse et al., Science 246:1275-1281 (1989)) to allow rapid and effective identification of monoclonal Fab fragments with the desired specificity for the target. Antibody fragments can also be produced by techniques in the art including, but not limited to: (a) a F(ab′)2 fragment produced by pepsin digestion of an antibody molecule; (b) a Fab fragment generated by reducing the disulfide bridges of an F(ab′)2 fragment, (c) a Fab fragment generated by the treatment of the antibody molecule with papain and a reducing agent, and (d) Fv fragments.

### Treatment Methods

Methods are provided for treatment or prevention of a disease or disorder associated with lung inflammation, such as COPD or lung inflammation caused by cigarette smoke, bacterial infection, viral infection, or a combination thereof. Diseases or disorders associated with lung inflammation include asthma, cystic fibrosis, bronchiectasis, and inflammatory bowel (IBD) related diseases.

As described herein, the present invention demonstrates that significant phenotypic changes can occur within the lung ILC population of COPD patients or those patients having disorders associated with lung inflammation. The present invention further demonstrates that the lung ILC population of COPD patients or those having lung inflammatory conditions can be characterized by a loss of GATA-3 and a decrease in the expression of Th2-associated markers, which strongly correlates with a marked expansion of IL-18Rα+T-bet⁺ ILC1s. Further, the present invention also provides that ILC1 expansion can be caused by a direct conversion from resident lung ILC2s which occurs in response to IL-12 and IL-18 stimulation. It has further been shown that a wide variety of triggers, especially those associated with exacerbations of COPD, initiate this plasticity in local ILC2s, including a number of viral and bacterial infections as well as cigarette smoke exposure.

In view of these observations, methods are provided herein for inhibiting the conversion of innate lymphoid cells (subset 2) (ILC2s) into innate lymphoid cells (subset 1) (ILC1s) where ILC2s are contacted with a modulator that prevents the switch of ILC2s to T-bet+ IFNγ+ ILC1s, maintains or suppresses the level of IL-12 receptor and/or IL-18 receptor expression in ILC1s, and/or maintains or increases the level of ST2, CRTH2 and/or GATA3 expression in ILC2s. Inhibition of the conversion of a population of ILC2s into ILC1s may facilitate prevention, treatment, including treatment to limit exacerbation of symptoms associated with COPD or underlying lung inflammatory conditions. Furthermore, such inhibition of ILC2s into ILC1s may limit the progression of COPD or limit the progression of other lung inflammatory conditions.

In order to provide more targeted or enhanced treatment options or treatment regimens for patients, additional methods are also provided where prevention, treatment, including treatment to limit exacerbation of symptoms is performed for a subject or patient who has been determined to have elevated levels of ILC or elevated ratios of ILC1s/ILC2s, where such determination is made by identifying a switch between a molecular signature of ILC2 to a molecular signature of ILC1, prior to administering a modulator or disease-modifying medication.

The present invention also provides that a molecular signature of ILC2 of the present invention corresponds to the expression of one or more Th2-associated transcripts selected from the group consisting of Gata3, Rora, Il4, Il5, Il9, Il13, Penk (proenkephalin), Areg (amphiregulin), Il17rb, and Il1rl1. A molecular signature of ILC1 of the present invention corresponds to a lower expression of one or more Th2-associated transcripts according to claim 26 and higher levels of one or more transcripts selected from the group consisting of Tbx21, Ifng, Il12rb2, Il18r1, Cxcr3, and Ccr5, wherein said levels are compared to the level of said transcripts in ILC2.

In one embodiment, the methods described herein comprise administering a modulator or a disease-modifying medication to a subject. Clinical response to administration of a modulator or disease-modifying medication can be assessed using screening techniques such as magnetic resonance imaging (MRI), x-radiographic imaging, computed tomographic (CT) scan, flow cytometry or fluorescence-activated cell sorter (FACS) analysis, histology, gross pathology, and blood chemistry, including but not limited to changes detectable by ELISA, ELISPOT, RIA, chromatography, and the like. Further, the subject undergoing therapy with a modulator or disease-modifying medication can experience improvement in the symptoms associated with the disease or disorder.

Methods of preparing and administering a modulator or disease-modifying medication to a subject in need thereof are well-known to or can be readily determined by those skilled in the art. The route of administration can be, for example, oral, parenteral, by inhalation, or topical. The term “parenteral” as used herein includes, e.g., intravenous, intraarterial, intraperitoneal, intramuscular, subcutaneous, rectal, and vaginal administration. Oral dosage forms include, e.g., capsules, tablets, aqueous suspensions, and solutions. Nasal aerosol or inhalation dosage forms can be prepared, for example, as solutions in saline, employing benzyl alcohol or other suitable preservatives, absorption promoters to enhance bioavailability, and/or other conventional solubilizing or dispersing agents.

Usually, a suitable pharmaceutical composition can comprise a buffer (e.g.

acetate, phosphate or citrate buffer), optionally a surfactant (e.g. polysorbate), optionally a stabilizer agent (e.g. human albumin), etc. The form and character of the pharmaceutically acceptable carrier or diluent can be dictated by the amount of active ingredient with which it is to be combined, the route of administration and other well-known variables. In one embodiment, the administration is directly to the airway, e.g., by inhalation or intranasal administration.

As discussed herein, a modulator or disease-modifying medication can be administered in a therapeutically effective amount for the in vivo treatment of a disease or disorder associated with lung inflammation. In this regard, it will be appreciated that the inhibitor(s) can be formulated so as to facilitate administration and promote stability of the active agent.

Pharmaceutical compositions in accordance with the present invention can comprise a pharmaceutically acceptable, non-toxic, sterile carrier such as physiological saline, non-toxic buffers, preservatives and the like. Suitable formulations for use in the therapeutic methods disclosed herein are described in Remington's Pharmaceutical Sciences, 20th ed., Mack Publishing Co., Easton, Pa. (2000).

The composition can be administered as a single dose, multiple doses, or over an established period of time in an infusion. Dosage regimens also can be adjusted to provide the optimum desired response (e.g., a therapeutic or prophylactic response). The amount of a modulator or a disease-modifying medication that can be combined with carrier materials to produce a dosage form will vary depending upon many different factors, including means of administration, target site, physiological state of the patient (i.e., the severity of the disease, the history of the disease, and the age, height, weight, health, and physical condition of the individual undergoing therapy), whether treatment is prophylactic or therapeutic, other medications administered, and whether the patient is human or an animal. Usually, the patient is a human, but non-human mammals, including transgenic mammals can also be treated. The amount of a modulator or disease-modifying medication to be administered is readily determined by one of ordinary skill in the art without undue experimentation, given this disclosure. Treatment dosages can be titrated using routine methods known to those of skill in the art to optimize safety and efficacy.

This disclosure also provides for the use of modulator or disease-modifying medication to treat or prevent a disease or disorder associated with lung inflammation, such as COPD or lung inflammation caused by cigarette smoke, bacterial infection, viral infection, or a combination thereof.

This disclosure also provides for the use of modulator or disease-modifying medication in the manufacture of a medicament for treating or preventing a disease or disorder associated with lung inflammation, such as COPD or lung inflammation caused by cigarette smoke, bacterial infection, viral infection, or a combination thereof.

A “disease-modifying medication” can be a bronchodilator, including a short-acting or long-acting bronchodilators, inhaled steroids, combination inhalers, oral steroids, phosphodiesterase-4 inhibitors, or theophylline.

All of the references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention. Documents incorporated by reference into this text are not admitted to be prior art.

### EXAMPLES

Embodiments of the present disclosure can be further defined by reference to the following non-limiting examples, which describe in detail preparation of certain antibodies of the present disclosure and methods for using antibodies of the present disclosure. It will be apparent to those skilled in the art that many modifications, both to materials and methods, can be practiced without departing from the scope of the present disclosure.

### Example 1. Lung-Resident ILC2 Downregulate GATA-3 Following Influenza Challenge

ILC2s are the major pulmonary ILC population, and are characterized by their expression of GATA-3. It has previously been shown that cigarette smoke silences lung-resident ILC2 responses by suppressing IL-5 and IL-13 production in these cells. The present invention provides relevance of this change, and whether other pathogenic stimuli associated with COPD could do the same.

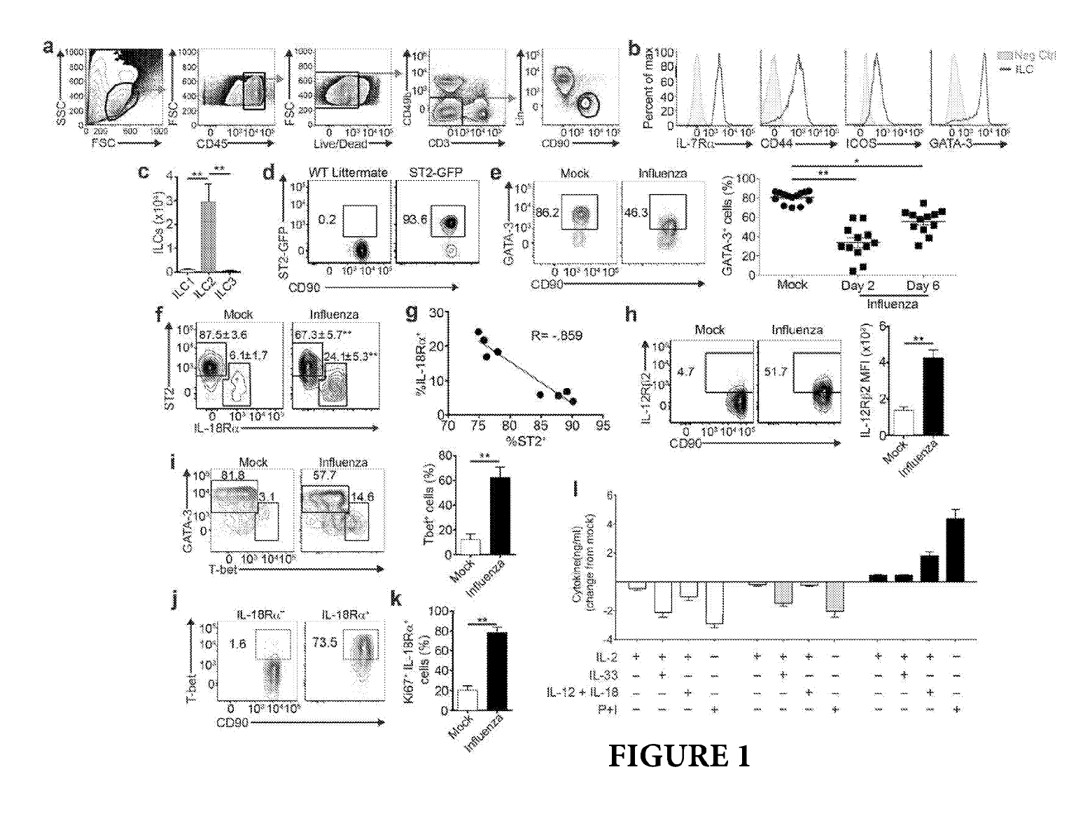

Lung ILC2s were defined as a population of non-T/NK cells, which were negative for expression of lineage markers and positive (high) for CD90, IL-7Rα, CD44, ICOS, GATA-3 and ST2 (FIG. 1a, 1b). The expression of ST2 on lung-resident immune cells is dynamically regulated following cigarette smoke exposure. Therefore, in the absence of specific reagents for ST2 and the need to more accurately understand its regulation, an ST2-GFP⁺ reporter mouse was developed. Although there were small populations of Lin⁻ CD90⁺ cells in naïve lung expressing T-bet or RORγt (i.e., ILC1 and ILC3 cells), the majority of ILCs were ST2^{HI} GATA-3^{m}, and represented approximately 85-90% of the total ILC pool. This observation was confirmed when assessing the ILC2s in the ST2 reporter mouse (FIG. 1c, 1d) where ILC2s were readily identifiable as Lin− ST2-GFP⁺ cells expressing CD90, IL-7Rα, CD44 and CD25.

Interestingly, infection with influenza A resulted in a rapid and dramatic loss (≥50%) of GATA-3 when compared to ILC2s from uninfected mice. This loss was observed as early as 48 h post infection (p.i.) and still evident at 6 days p.i. (FIG. 1e). Further, loss of GATA-3 correlated with a marked reduction in the expression of the ILC2-associated marker ST2 in these cells following infection (FIG. 1f). The loss of these markers was not associated with ILC2 egress or cell death, since there was no detection of significant ILCs in the blood, draining lymph nodes (dLN), or bronchoalveolar lavage (BAL); and, the ILCs in the lung did not appear to be apoptotic, as determined by PI and Annexin V staining.

Since viral infection is associated with the production of pro-inflammatory cytokines, including IL-12 and IL-18, the receptor expression for these Th1 associated mediators in ILCs was examined. Strikingly, the loss of ST2 on ILC2s strongly correlated with a parallel increase of an IL-18Rα⁺ ILC population (FIG. 1f, 1g; R=−0.859). Also observed was a significant increase in IL-12Rβ2 on this IL-18Rα⁺ ILC population following infection, (FIG. 1h), although IL-12Rβ2 expression did not appear to correlate with the loss of ST2, suggesting that IL-12Rβ2 may appear earlier than IL-18Rα on these cells.

T-bet is a transcription factor downstream of IL-12 that is a marker of ILC1s and is tightly linked with the promotion of Th1 responses. Lung-resident ILCs for T-bet expression were therefore examined, and loss of GATA-3 in these cells occurred concomitantly with an increase in the percentage of ILCs expressing T-bet at day 7 p.i. was found (FIG. 1i). Indeed, virus-induced T-bet⁺ ILCs were all contained within the IL-18Rα⁺ subset in the lung (FIG. 1j). While the IL-18Rα+T-bet⁺ ILCs remained CD3⁻, CD49b⁻, Lin⁻, CD90⁺ and CD44⁺, they expressed intermediate levels of CD25, IL-7Rα, ICOS and c-Kit, compared to ST2⁺ ILCs.

Interestingly, the change in ILC populations following viral infection appeared to be specific to an ILC1-like response, as significant RORγt⁺ ILC3s in the lung at these same timepoints was not detected. Moreover, the majority of proliferating Ki67⁺ ILCs in the lung during infection expressed the IL-18Rα⁺ (FIG. 1k), suggesting a role for IL-18Rα in the maintenance or function of these cells.

To examine the consequences of these changes in transcription factor expression, total lung resident ILCs were isolated from influenza infected mice and stimulated ex vivo to assess cytokine expression patterns. It was found that lung ILCs from infected mice produced markedly less IL-5 and IL-13 at day 7 p.i. than ILCs from mock (PBS treated) animals (FIG. 1l). However, consistent with the up-regulation of receptors for IL-12 and IL-18, ILCs isolated from the lungs of infected animals now produced significant levels of IFN-γ in response to IL-12 and IL-18 stimulation ex-vivo (FIG. 1l).

Collectively, these data demonstrate a major change in both the phenotype and functional ability of the resident lung ILCs during viral challenge, characterized by a decrease in local ILC2 function and an expansion of IL-18Rα+T-bet⁺ IFNγ⁺ ILC1s.

### Example 2. Cigarette Smoke Exposure and Bacterial Infection Induce Phenotypic Changes in Lung ILCs

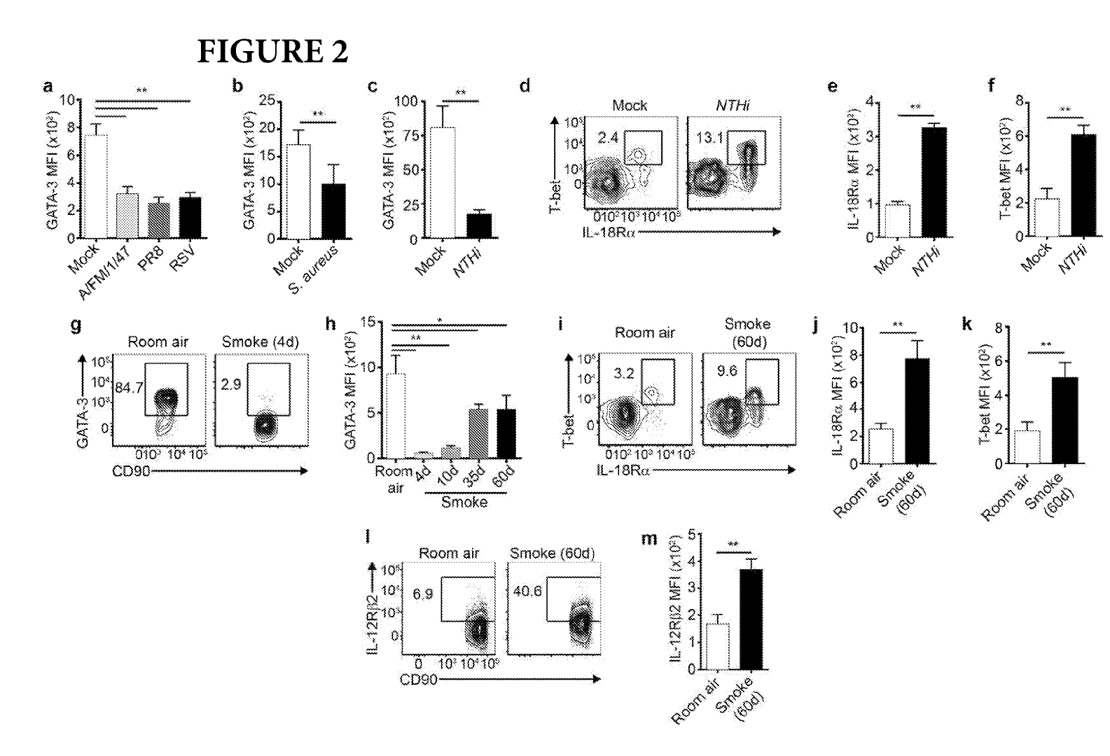

Since exacerbations of COPD are associated with a number of viral and bacterial respiratory infections, whether the switch in ILC subgroups occurred in mice challenged with different COPD-relevant pathogens was investigated. Strikingly, a very similar pattern was found. Inoculation with a different strain of influenza A (PR8), respiratory syncytial virus (RSV) (FIG. 2a), or the Gram-positive and -negative bacteria, Staphylococcus aureus, (FIG. 2b), and non-typeable Haemophilus influenzae (NTHi), (FIG. 2c-2f) all induced the loss of GATA-3, and the subsequent increase in IL-12Rβ2, IL-18Rα, and T-bet expression within the lung ILC population.

This shift in ILC subgroups was not limited to infectious triggers in that cigarette smoke exposure, one of the primary causative factors for COPD, induced a dramatic loss of GATA-3 (i.e., after 4 and 10 days of smoke inhalation). GATA-3 levels remained significantly reduced through 6 months of continuous smoke exposure (FIG. 2g-2h; data shown up to 8 weeks). Notably, the emergence of an IL-12Rβ2⁺, IL-18Rα⁺, T-bet⁺ ILC1 population was also observed after 8 weeks of smoke exposure (FIG. 2i-2m) and was maintained after 6 months of further treatment. Finally, in a COPD exacerbation model, where cigarette smoke exposure is combined with viral infection, the changes in lung-resident ILCs were found to be dramatically augmented. Mice previously exposed to cigarette smoke had greater suppression of GATA-3 and ST2 and much higher levels of the IL-18Rα in lung-resident ILCs, compared to smoke or virus alone.

Taken together, these data demonstrate that the phenotypic switch in the local ILC population may be a general mechanism in response to a foreign and/or environmental insult.

### Example 3. IL-12 and IL-18 Induce IFN-γ Producing ILC1s from Local ST2+ ILC2 Pool

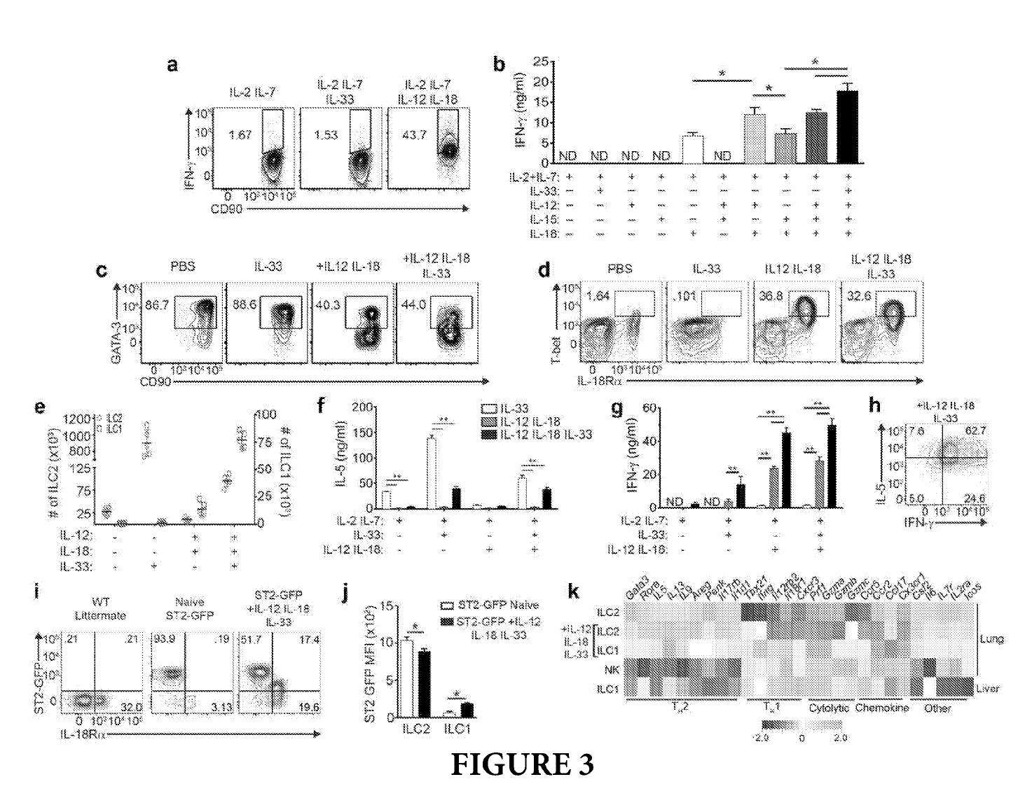

To examine whether ILC2s could adopt an ILC1 phenotype, IL-33-expanded ILC2s were enriched to >98% purity from the lung and cultured with various cytokines for 4-7 days. Stimulation of these cells with IL-12 or IL-15 did not induce the production of IFN-γ from ILC2s. However, in response to IL-18, and in particular, the combination of IL-12 and IL-18, ˜20-50% (respectively) of these cells produced IFN-γ. This was also reflected in the levels present in culture supernatants (FIG. 3a, 3b). These data are consistent with studies demonstrating that IL-12 and IL-18 can induce IFN-γ production from ILC3s.

Despite previous reports that IL-15 promotes expression of IFN-γ by ILC1s, it was found that IL-15, alone or in combination with IL-12 and/or IL-18, did not significantly affect IFN-γ production under these conditions (FIG. 3b). Production of IL-5 was remarkably consistent across different conditions in this assay, which supports the notion that ILC2 constitutively produce this cytokine. Although IL-13 production was more variable, levels were consistently higher in response to NFκB-activating cytokines, particularly IL-33 and IL-18. Strikingly, ILC2s co-expressed IL-13 and IFN-γ when stimulated with IL-12 and IL-18, demonstrating that these cells have promiscuous patterns of cytokine expression that is consistent with functional plasticity of ILC2s. Thus, lung-derived ILC2s can express ILC1-associated markers and produce abundant levels of IFN-γ in response to IL-12+ IL-18.

To determine whether IL-12 and IL-18 could induce ILC1 expansion in vivo, ILC responses following intranasal administration of IL-12+ IL-18, IL-33 alone, or the combination of all three cytokines were examined. Local co-administration of IL-12p′70 and IL-18 to naïve mice significantly decreased expression of GATA-3 and ST2 in total lung-resident ILCs compared to PBS or IL-33-treated controls, (FIG. 3c), and was similar to that observed during viral infection (FIG. 1). Furthermore, IL-12+ IL-18 treatment markedly increased the expression and number of IL-18Rα⁺, T-bet⁺ ILCs, which produced dramatically less IL-5 and IL-13, but significantly higher quantities of IFN-γ ex vivo, when compared to PBS or IL-33 treatment (FIG. 3d-3g).

Intranasal delivery of IL-33 significantly increased the numbers of ST2+, GATA-3⁺ ILC2s within the lung, which produced high levels of IL-5 (FIG. 3f) and IL-13. Interestingly, despite the marked increase in the proportion of ILC1s, in response to IL-12+ IL-18 (FIG. 3c, 3d), a minimal, yet significant, increase in the total number of these cells was observed (FIG. 3e). However, administration of IL-33 in combination with IL-12+ IL-18 dramatically increased the total number of ILC1s at the expense of ILC2 expansion (FIG. 3e). Moreover, the ability of IL-33 to induce production of Th2 cytokines was markedly abrogated in the presence of IL-12 and IL-18, and these cells produced significant quantities of IFN-γ (FIG. 3g). These cells also co-expressed IL-5 and IFN-γ (FIG. 3h), consistent with the emergence of an IL-18Rα⁺ T-bet⁺ IFN-γ⁺ ILC1 from the local ILC2 pool. Notably, administration of either IL-12 or IL-18 alone had minimal effect on expression of GATA-3, T-bet, IL-18Rα or IFN-γ. Thus, IL-12 and IL-18 co-regulate ILC1 expansion in vivo, while IL-33 acts in a context-dependent manner to amplify this response.

ST2 is a direct target of GATA-3 in ILC2s. To determine whether ILC1s expand from the local ILC2 pool following cytokine treatment, these experiments in ST2 GFP⁺ mice were repeated. A comparison of ST2-GFP⁺ versus IL-18Rα expression revealed two distinct populations of ILCs in naïve lung, ST2-GFP+ IL-18Rα⁻ ILC2s (93.4%) and ST2-GFP⁻ IL-18Rα⁺ ILC (4.01%), which were dramatically altered upon IL-12+ IL-18+IL-33 treatment, resulting in an increased frequency of ILC1s, as determined by their expression of IL-18Rα (FIG. 3i). Strikingly, these IL-18Rα+ cells were also intermediate for ST2-GFP (FIG. 3i), and estimated to represent approximately 50% of the original ST2-GFP+ ILC2 population. Ad hoc analysis of GFP⁺ mean fluorescence intensity (MFI) revealed moderate yet significant decreases in ST2 expression on ILC2s following cytokine treatment. The expression of ST2-GFP⁺ on IL-18Rα+ cells was lower, albeit markedly still increased, compared to ST2-GFP⁻ IL-18Rα⁺ ILC1s in naïve lung (FIG. 3j). Thus, ILC1s emerge from the local ST2+ ILC2 pool, rather than from the outgrowth of pre-existing, bona fide, ILC1s within the lung.

Comparison of gene expression profiles among ILC2s sorted from naïve mice versus ILC2s (ST2⁺ IL-18Rα⁻) and ILC1s (ST2⁻ IL-18Rα⁺) sorted from IL-12+ IL-18+IL-33 (cytokine) treated mice revealed partially overlapping but distinct molecular patterns. ILC2s isolated from naive lung expressed Th2-associated transcripts, including Gata3, Rora, Il4, Il5, Il9, Il13, Penk (proenkephalin), Areg (amphiregulin), Il17rb, and Il1rI1. Similarly, ILC2s from cytokine-treated mice up-regulated many of these same genes (FIG. 3k). In contrast, the IL-18Rα+ ILC from cytokine treated animals appeared to have an altered, unique gene expression profile, characterized by lower expression of the aforementioned ILC2 transcripts and higher levels of Tbx21, Ifng, Il12rb2, Il18r1, Cxcr3, and Ccr5 (FIG. 3k). Notably, these cells were intermediate in phenotype between naïve liver ILC1s/lung NK cells, versus cytokine expanded ILC2s, as their Th1-associated transcripts were higher but they expressed markedly lower levels of genes associated with cytolytic function (e.g., Prf1, Gzma, Gzmb, Gzmc) (FIG. 3k). Interestingly, also observed were differences in the subsets of ILCs from cytokine-treated mice regarding chemokine receptors, e.g., Ccr5, Ccl17, and Cx3cr1, while the expression of Il7ra, Il2r, and Icos was lower in activated ILC1s when compared with ILC2 (FIG. 3k).

Collectively, these data highlight that ILC2 plasticity involves a switch within the molecular signatures, which concurs with the proteomic changes reported via flow cytometry.

### Example 4. ILC2s Directly Convert to ILC1s During Viral Challenge and Cluster within Areas of Inflammation

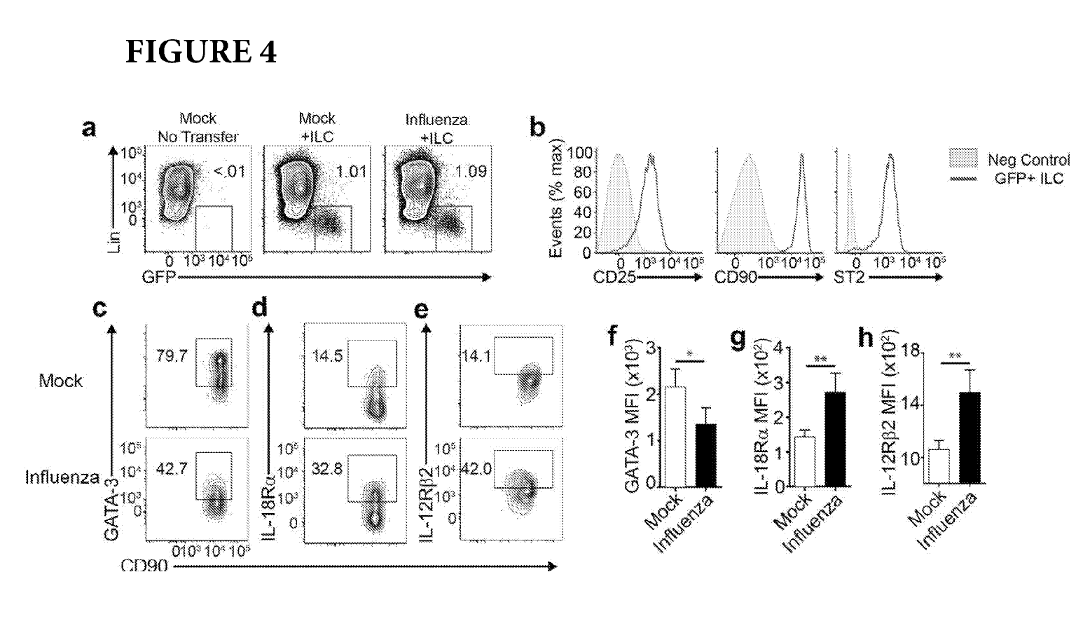

Whether ILC2s can convert and/or have the capacity to switch to ILC during infection were directly tested using RAG/γc double knockout mice, which lack ILCs, NK cells, and mature lymphocytes. Lung resident ST2^{HIGH}, IL-18Rα⁻ ILC2s were FACS-sorted from IL-33-treated GFP⁺ transgenic mice and transferred intravenously into RAG/γc^{-/-} mice 12 h prior to influenza A infection (FIG. 4a). IL-33-expanded GFP+ ILC2s expressed high levels of GATA-3 and Th2 cytokines, but not ILC1 markers (FIG. 4b-4f). This protocol was used to ensure that all ILCs would be readily identifiable by GFP expression.

The response to infection was delayed and lethal in RAG/γc^{-/-} animals compared to wild type mice; therefore, to prolong survival these mice were reconstituted (intraperitoneally) with GFP⁻ splenocytes and lung-derived NK and T cells at the time of infection. Transferred GFP⁺ ILCs were then analyzed at 7 and 10 days p.i for changes in their phenotypic profile, including expression of GATA-3, IL-12Rβ2 and IL-18Rα. Upon adoptive transfer, GFP+ ILCs infiltrated the lungs of recipient mice, and were readily identifiable by flow cytometry in naïve and infected animals as Lin GFP cells expressing CD25, CD90, and ST2 (FIG. 4b).

Further, in response to infection, these GFP+ ILC2s had significantly down-regulated GATA-3 expression by day 7 p.i. (FIG. 4c, 4f). Moreover, this loss of GATA-3 correlated with a striking up-regulation of IL-18Rα and IL-12Rβ2 on these GFP+ cells (FIG. 4d, 4e, 4g, 4h). Also observed were similar phenotypic changes in GFP+ ILC2s at Day 10 p.i., however, T-bet expression at days 7 or 10 p.i was not detected, potentially due to the delayed response to infection in these mice. Remarkably, upon ex vivo stimulation with IL-12+ IL-18, the GFP+ cells were able to produce significant amounts of IFN-γ at 10 days p.i. and this change coincided with down-regulation of IL-13.

Strikingly, immunohistochemical (IHC) analysis of lung tissue from these same mice revealed a marked patchy-like response to infection within the lung. The transferred GFP+ ILCs, appeared to cluster within the inflammatory foci characteristically associated with virus-induced inflammation. Higher power magnification revealed that ILCs typically clustered within areas of viral replication, even in areas of the tissue closely bordering non-inflamed regions. However, GFP+ ILCs in non-inflamed regions of the parenchymal tissue were also observed, although these were much fewer in number and predominantly solitary. Using double IHC, the co-localization of these clusters of ILCs with influenza⁺ epithelial cells was confirmed, as well as perivascular regions neighboring the inflamed airways. Importantly, a combination of IHC and in situ hybridization revealed that myeloid-derived cells expressing IL-12 and IL-18 mRNA, were frequently identified in close proximity to GFP+ ILCs within the inflammatory areas of the lung.

Quadruple histochemistry was employed with combined immunofluorescence and chromogenic IHC (see Example 9) to examine and quantify GATA-3 associated ILC expression in the lung following infection. Using computerized image analysis of digitized whole slide images, all individual GFP ILCs (green fluorescence) were automatically identified in the tissue and scored on their staining intensity for blue DAPI, (a DNA marker for total nuclear content per cross section), as well as their red/pink GATA-3 immunostaining.

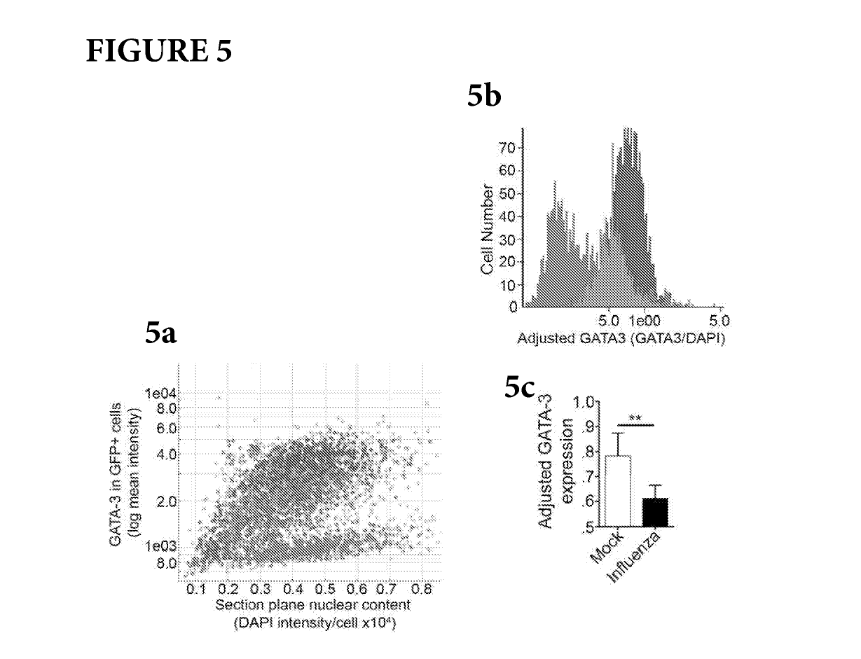

Analyses were performed on GATA-3^{HIGH} ILCs in a control mouse, and a non-infected region from an influenza-treated animal compared with typical GATA-3^{LOW} ILCs in infected areas at d5 and d6 p.i., respectively. ILC2-associated GATA-3 expression in whole lung sections from control (PBS treated) versus infected mice was first examined. Scattergram analysis of individual GFP cells clearly shows that viral-infected lungs contained significantly more GATA-3^{LOW} expressing ILCs compared to uninfected mice (FIG. 5a). After further confirmation of a uniform infection in all influenza treated mice, and since the nuclear representation differs in 2D cross sectioned cell areas, GATA-3 expression in each GFP cell was normalized to the cell's nuclear DAPI content. Again, a marked reduction in the frequency of GATA-3^{HIGH} ILCs in infected mice compared to controls was found (FIG. 5b).

Interestingly, a time dependent loss of GATA-3 in ILCs from influenza treated mice was observed, since GATA-3 intensity was further diminished in the GFP cells of mice examined at day 6 versus day 5 p.i. (FIG. 5a, 5b). FACS data confirmed this time course of GATA3 expression. Importantly, a statistically significant reduction in the ILC-associated GATA-3 levels between control and infected mice was quantified by comparing the mean GFP GATA-3 expression per individual mouse lung section (FIG. 5c; data from d5 p.i.).

Finally, given the uneven nature of the infection, the loss in GATA-3 expression was specific to the inflamed microenvironments throughout the lung tissue was assessed. Thus, using spatial data (x, y co-ordinates) from quadruple (influenza, GFP, GATA-3 and DAPI) stained sections, a focused analysis comparing GATA-3 expression levels in the areas of lung tissue with higher versus lower cellular and virus density was performed. Patchy cell-dense areas of the lung were compared with a virus density heatmap from the same section, as well as with images where the spatial distribution of individual GFP cells had been plotted and color-coded according to their GATA-3 intensity. Remarkably, GATA-3^{LOW} ILCs were much more frequent within the infected areas of the lung. In contrast, the ILCs found in the surrounding, uninfected tissue regions were predominantly GATA-3^{HIGH}.

In summary, these data demonstrate that ILC2 directly convert to an ILC1-like phenotype following viral challenge, and migrate to areas associated with viral replication. This is presumably a response to local pro-inflammatory cues, such as IL-12 and IL-18, which govern their subsequent plasticity.

### Example 5. T-Bet is Dispensable for Silencing ILC2 but Required for Production of IFN-γ

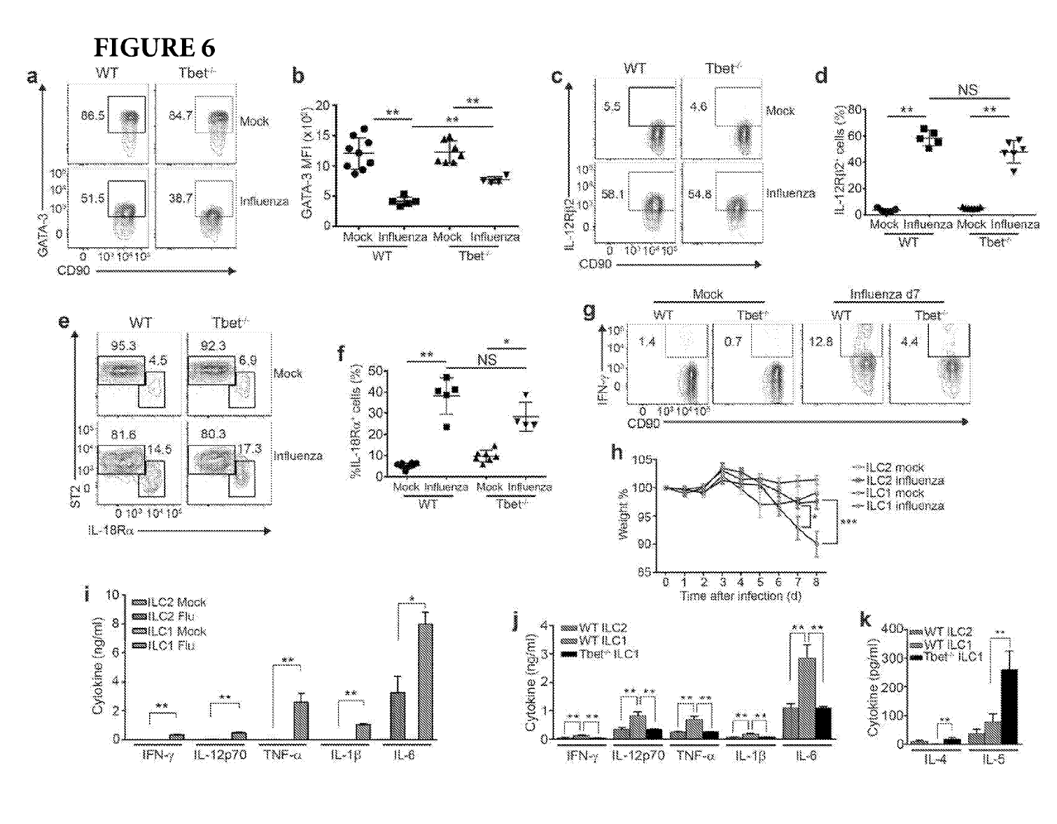

To determine whether there was a functional role for T-bet in the maintenance and/or proliferation of ILC following viral challenge, ILC responses in T-bet deficient mice were examined. Interestingly, T-bet was not required for the loss of GATA-3 and ST2 or the gain of IL-12Rβ2 and IL-18Rα expression on these cells, nor was T-bet required for ILC1 proliferation when compared to C57BL/6 (WT) mice (FIG. 6a-6f). However, ILCs isolated from infected T-bet^{-/-} animals were markedly impaired in their capacity to produce IFN-γ after ex vivo stimulation with IL-12+ IL-18 (FIG. 6g). Further, ILCs from WT and T-bet^{-/-} mice responded similarly to intranasal administration of IL-12+ IL-18 by down-regulating GATA-3 and ST2, and up-regulating the IL-12Rβ2 and IL-18Rα chains. However, as in the viral infection studies, T-bet was required for maximal expression of IFN-γ in response to IL-12+ IL-18. Thus, T-bet is dispensable for the conversion of ILC2 into ILC1s in response to Th1-type inflammation, but appears to be essential for optimal production of IFN-γ in these cells.

### Example 6. ILC1s Are Pathogenic and Dramatically Amplify Virus-Induced Weight Loss and Inflammatory Responses to Infection

The biological relevance for ILC2 plasticity and their specific phenotypic switch to ILC during infection was next examined. However, given the lack of ILC-specific surface markers, there are limited strategies for antibody-mediated depletion of ILC populations. Antibodies directed against the Thy antigen (CD90) have been used successfully to deplete tissue-resident ILCs, in animals devoid of adaptive immune cells. While the fact that treatment with anti CD90 antibody depletes >90% of lung-resident ILCs was confirmed, it was also found that this antibody systemically depleted ˜70% of NK cells, as well as a population of lung-resident CD90⁺ CD45⁻ CD166⁺ Sca-1⁺ mesenchymal stem cells.

Therefore, in order to more clearly address the biological function of ILC1s, an adoptive transfer of ILC or ILC2s in immunocompromised mice was performed (FIG. 6h). Lung resident ILC2s (ST2⁺, GATA-3⁺) and ILC1s (IL-18Rα⁺, T-bet⁺) were isolated from IL-33 or IL-12+ IL-18+IL-33 treated C57BL/6 mice and transferred into C57BL/6 RAG/γc deficient mice, 24 h prior to influenza A infection. Strikingly, animals that received adopted ILC1s exhibited significantly more weight loss to infection compared to mice that were reconstituted with ILC2s prior to infection (FIG. 6h). Moreover, transfer of ILC correlated with a dramatic, exaggerated, pro-inflammatory cytokine production, including IFN-γ, IL-12p′70, TNFα, IL-1α, and IL-6 (FIG. 6i).

Differences in cellular inflammation following ILC1 transfer at this time point were not observed, presumably due to the delayed response to infection in RAG/γc deficient mice. To definitively test whether T-bet/IFN-γ was required for the inflammatory potential of ILC1s, the adoptive transfer was repeated using ILC1s isolated from C57BL/6 (WT) and T-bet deficient mice. As previously shown (FIG. 6i), ILC1 transfer was associated with exaggerated Th1 cytokine production (FIG. 6j); however, T-bet deficient ILC1s were unable to promote this augmented, anti-viral, response, including IFNγ production (FIG. 6j). Strikingly, the absence of T-bet in these cells significantly triggered Th2 cytokine production, i.e., IL-4 and IL-5, within the lung. (FIG. 6k).

Thus, IL-12 and IL-18 expand a population of ILC1s with the capacity to dramatically augment virus-induced inflammation, while T-bet is required for the full inflammatory potential of these cells.

### Example 7. IL-12 Induces Plasticity in Human ILC2s

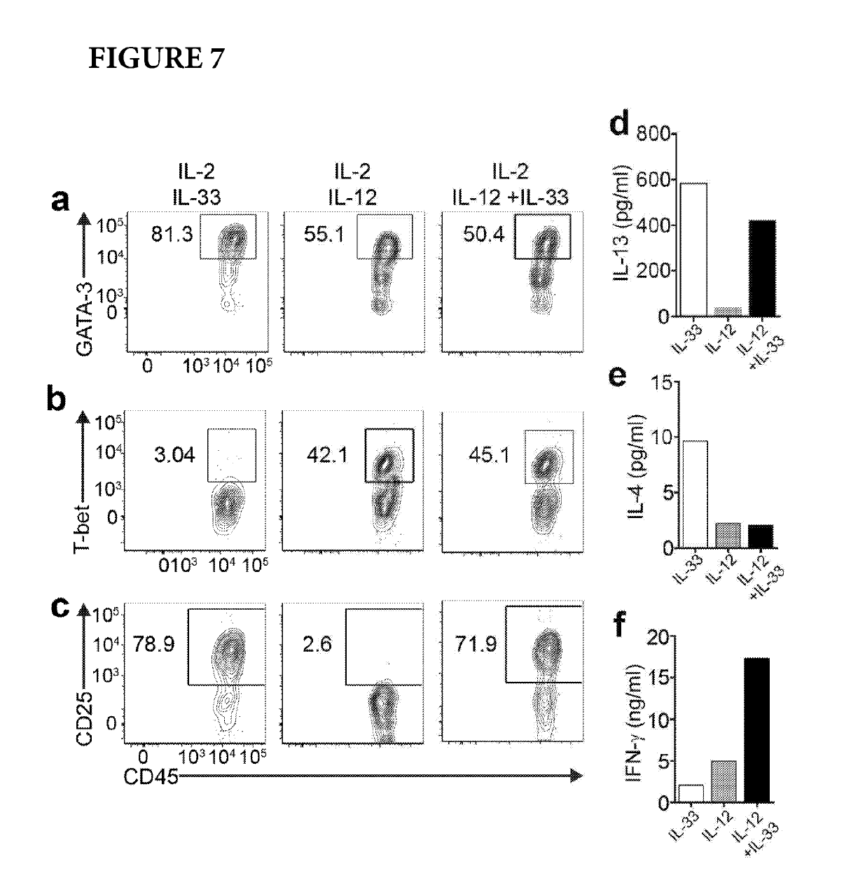

To test whether human ILC2 can demonstrate plasticity and differentiate into ILC1s, Lin⁻ IL-7Rα⁺ CRTH2⁺ CD161⁺ ILC2s were sorted from the peripheral blood of healthy donors, cultured for 5 days in the presence of IL-2+ IL-33 or IL-2+ IL-12, and examined for surface markers and cytokine output. As previously reported, it was found that CRTH2 is a marker for human ILC2s, and ILC2s cultured in IL-2+ IL-33 were CRTH2+, GATA3⁺, Tbet⁺, CD25⁺, CD161⁺ and IL-7Rα⁺ (FIG. 7a-7c). These cells produced high levels of IL-13 and IL-4 but significantly lower amounts of IFN-γ (FIG. 7d-7f). Notably, freshly isolated peripheral blood ILC2s produced little or no IL-5 in response to IL-33, consistent with the observations of Ohne et al. In contrast, the addition of IL-12 to the culture media significantly reduced levels of GATA-3 and CD25 in ILC2s, but concomitantly prompted a dramatic increase in T-bet expression in these same cells (FIG. 7a-7c), corroborating observations in mouse ILC phenotypes. Further, in response to IL-12, these ILC2s produced lower levels of IL-13 and IL-4, even in the presence of IL-33 (FIG. 7d, 7e). Moreover, IL-12 induced high levels of IFN-γ in human ILC2s and these levels were augmented in the presence of IL-33 (FIG. 7f).

Thus, human ILC2s can acquire an ILC1 phenotype following IL-12 stimulation, and, analogous to mouse ILC2s, IL-33 can amplify the production of IFN-γ by these cells.

### Example 8. COPD Patients have Dramatically Altered ILC Responses that Correlate with Disease Severity

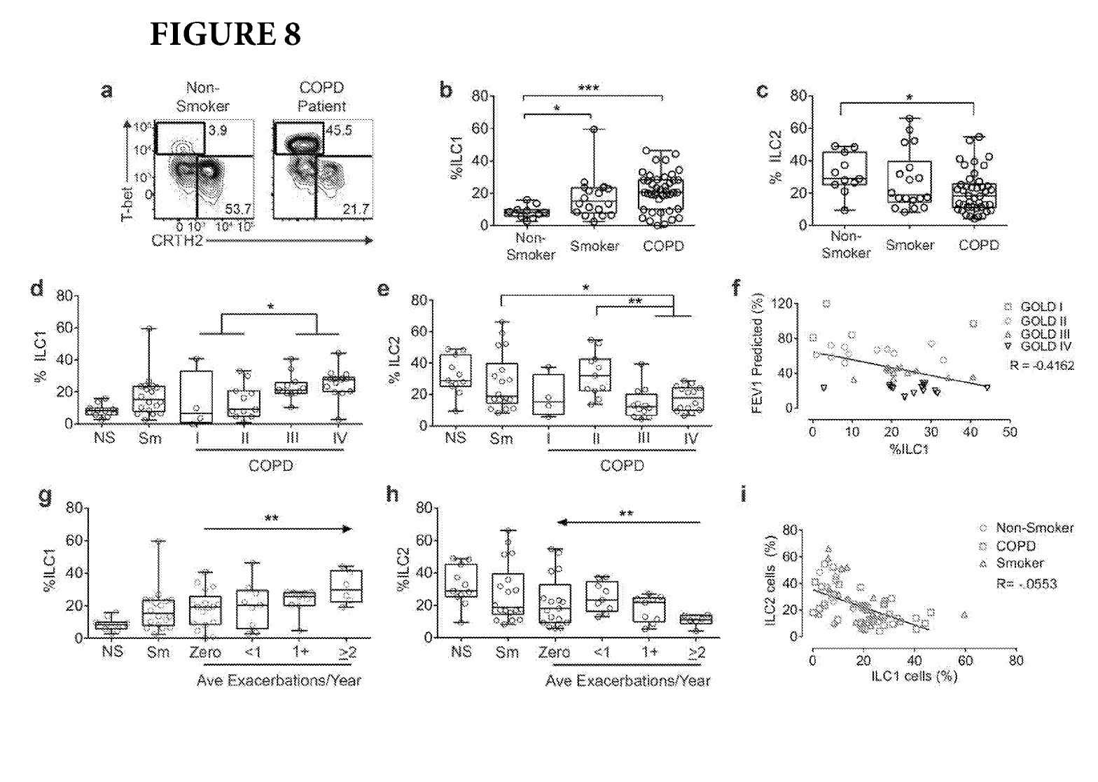

Individuals with COPD experience acute worsening of symptoms following infection with viral or bacterial pathogens and these exacerbations correlate with patient morbidity and mortality. Mouse models of COPD developed and utilized for the present invention demonstrate significant functional and phenotypic changes in lung-resident ILC populations following exposure to virus, bacterial infection, or cigarette smoke alone, and these effects are exaggerated when smoke is combined with viral infection (FIG. 2). To investigate whether similar changes occurred in COPD patients, the frequencies of ILC subsets in the peripheral blood of stable patients from the COPDGene cohort were examined. Analysis of circulating ILCs in the peripheral blood of healthy controls revealed that ˜40% of circulating ILCs expressed the ILC2 marker CRTH2, as well as CD25, IL7Rα, and GATA-3, and ˜5% of circulating ILCs were ILC (defined here as T-bet^{HI}, FIG. 8a), which is consistent with previous reports. It was found that many ILCs in circulation do not express known ILC1, ILC2, or ILC3 markers, and it is unclear whether these represent circulating ILC precursors or immature cells.

It was observed that the frequency of ILC1s in COPD patients was markedly elevated in the circulation when compared to healthy controls (FIG. 8a, 8b). This increase in ILC correlated with a concomitant decrease in the frequency of circulating ILC2 cells (FIG. 8c). Moreover, patients with more severe disease (GOLD III/IV) had significantly higher frequencies of ILC1s when compared to patients with milder disease (GOLD I/II; FIG. 8d). Conversely, ILC2 ratios were markedly lower in the more severe patients (GOLD III/IV) compared to milder patients (GOLD II) or healthy smokers (FIG. 8e). Matched (age and smoking pack years) healthy smokers also exhibited significant increases in ILC1 ratios compared to healthy controls although there was an (apparent) trend for a higher frequency of ILC1s in all COPD patients (GOLD I-IV) compared to healthy smokers (FIG. 8b). Of interest, several individuals of the ‘healthy smoker” group presented with relatively low FEV1/FVC ratios, (0.73-0.71); i.e., similar to the ratios found in milder COPD patients (≤0.7), which may account for the increase in ILC1s in some of these subjects. Nevertheless, there was a significant inverse correlation between the % ILC1s and lung function, as assessed by FEV1% predicted (R=−0.4162, p<0.01) and FEV1/FVC ratio (R=−0.4154, p<0.01), (FIG. 8f). In contrast, the % ILC2s in COPD patients positively correlated with lung function parameters.

Of importance, there was a significant linear trend between the % ILC and the average number of exacerbations, in that patients with ≥2 exacerbations/year had the highest ILC1 and the lowest ILC2 frequencies (FIG. 8g, 8h). Consistent with the reciprocal relationship between the % ILC1s versus ILC2s, and the disease-relevant clinical endpoints, a strong correlation (R=−0.5529, p<0.001) between the ratio of ILC1 versus ILC2 was found, thus supporting the hypothesis for direct conversion of ILC2s to ILC1s in COPD (FIG. 8i). Indeed, inverse correlations between ILC1 and ILC2 were consistently observed; yet the sum of ILC1 and ILC2 remain at the same level (42.3±13.1%; mean±SD), suggesting this could be attributable to plasticity within the existing ILC population.

Collectively, these data demonstrate that COPD is associated with marked changes in the pool of circulating ILCs and that increases in the frequency of ILC1s correlate with disease severity, lung function and frequency of exacerbations.

### Example 9. Materials and Methods

### Mice

BALB/c (Harlan), SCID (Jax), C57BL/6 (Jax), RAG/SCID deficient (Taconic), GFP transgenic (C57BL/6-Tg(CAG-EGFP)131Osb/LeySopJ, Jax) mice were housed at MedImmune and treated according to protocols approved by the IACUC. T-bet −/− (Jax) mice were housed and maintained at the University of Pennsylvania according to institutional guidelines and protocols.

### Generation of ST2-GFP Reporter Mice

A targeting construct was injected into murine blastocysts. The construct contained an upstream 4kb short homology arm, followed by an FRT-flanked puromycin resistance cassette nestled in intron 10, then exon 11 of the Il1rl1 gene, where the stop codon was deleted, and immediately followed by a fusion sequence of the T2A self-cleaving peptide fused to eGFP, followed by a new stop codon. The construct ended with a 6kb long homology arm. All sequences used to create the targeting construct were based on the Ensembl transcript ENSMUST00000097772 (Il1r11-001), which corresponds to Il1rl1 Isoform A (Membrane-bound; Uniprot ID: P14719-1). After Flp-mediated recombination, the puromycin selection gene was deleted, leaving a single FRT site remaining in intron 10 of the targeted Il1rl1 gene, followed by the exon11-T2A-eGFP fusion sequence. All ST2-reporter mice used for experiments were heterozygous ST2^{+/GFP} on a BALB/c background. Littermates were used as controls.

### Smoke Models

Cigarette smoke exposed mice were smoked twice a day, 5 days a week following previously published protocols and analyzed at timepoints specified in FIG. 2.

### Bacterial and Virus Challenge Models

50XTCID50 of influenza A (A/FM/1/47), 50XTCID50 PR8 strain (both are H1N1), or 10⁶ pfu RSVA2 were administered to mice intranasally. In some studies, influenza A was administered after smoke exposure according to previously established protocols. Mice were analyzed at timepoints specified in the figures. Mice were treated intranasally with 10⁷ cfu of non-typeable Haemophilis influenzae and analyzed at day 2 and day 5 post-infection, or with 10⁵ cfu of Staphylococcus aureus and analyzed at day 5 post-infection.

### In Situ ILC Expansion

To expand ILC populations for in vitro experiments, BALB/c SCID mice were treated intranasally with 1 μg of recombinant IL-33 (generated in-house), IL-12 (eBiosciences), or IL-18 (R&D) at D1, D3, and D5, and sacrificed at D7. ILCs were enriched as described below for ex vivo analysis. To expand ILC2, IL-33 was used. For ILC1 expansion, IL-12+ IL-18+IL-33 were used.

### Fluidigm Analysis

ILC2s (defined as CD45+ viable CD3− CD49b− Lin− CD90+ ST2+ IL-18Rα−) were sorted from naïve lung. ILC2s (CD45+ Viable CD3− CD49b− Lin− CD90+ ST2+ IL-18Rα−) and ILC1s (CD45+ Viable CD3− CD49b− Lin− CD90+ ST2− IL-18Rα+) were sorted from lungs of mice treated with IL-12+ IL-18+IL-33 as described above. For controls, NK cells (CD45+ Viable CD49b+CD3−) were sorted from naïve lung, and ILC1s were sorted from naïve liver (CD45+ Viable CD3− CD19− NKp46− CD11a+ TRAIL+). RNA was isolated from cells, and qPCR was performed using the Fluidigm Biomark Dynamic array, loaded with probes for transcripts of interest (Fluidigm Corp., South San Francisco, Calif.).

### ILC Enrichment (Mouse)

Lungs were perfused with PBS, diced into ˜2 cm pieces, and incubated with Liberase™ and DNAse (both Roche) for 45 minutes at 37° C. before being mashed through a 70 μm cell strainer and washed with complete RPMI. Remaining blood cells were lysed with ACK cell lysing buffer (Invitrogen), and single cell suspensions were incubated with biotinylated antibodies against CD3, CD19, B220, CD5, TCRαβ, TCRγδ, CD11c, F4/80, Gr1, Ter119, CD49b, and CD27. Cells were then incubated with anti-biotin microbeads (Milltenyi) and depleted following manufacturer's protocol. For each ILC enrichment, the depletion was repeated twice and typically yielded >95% pure ILC populations.

### ILC Isolation for In Vitro Culture (Human)

Blood was collected from healthy volunteers recruited by the MedImmune Blood Donor program. All volunteers gave informed consent. PBMCs were isolated using CPT tubes (BD) according to the manufacturer's protocol. Remaining red blood cells were lysed using ACK lysis buffer. PBMCs from 3-4 healthy donors were pooled and depleted using an NK cell enrichment kit (StemCell Technologies). ILCs were then sorted to 99% purity as viable, CD45+, nonT/nonB, Lin−, IL-7Rα+, CD161+, CRTH2+ cells.

### Ex Vivo Stimulation of ILCs

ILCs were isolated from murine lung or human blood as above, and stimulated with IL-2, IL-7, IL-33, IL-12, IL-18, IL-15, or combinations thereof, as stated in the figure. All cytokines were used at 50 ng/mL. Cells were analyzed by flow cytometry, and supernatants were assayed for cytokine production by MSD or ELISA (MesoScale Diagnostics).

### COPD Samples

Blood was collected from healthy controls, smoking controls, and COPD patients enrolled in the COPDGene cohort. Patient demographics are given in Table 1 and Table 2. All work was approved by the IRB at National Jewish Health.

TABLE 1

| | Non-Smoker Control | Smoker Control | COPD | |
|---|---|---|---|---|
| | (N = 10^{a}) | (N = 18^{a}) | (N = 42^{a}) | p-Value |
| Age | 62.20 ± 9.45 | 66.22 ± 10.52 | 68.21 ± 7.43 | 0.1625 |
| Gender (% Male) | 4 (40.00) | 5 (27.78) | 21 (50.00) | 0.2618 |
| BMI | 25.54 ± 3.62 | 30.34 ± 12.40 | 38.10 ± 56.75 | 0.1326 |
| Smoker (% Current) | 0 (0.00) | 4 (22.22) | 5 (11.90) | 0.2946 |
| FEV1 (%) | 99.00 ± 12.62 | 89.56 ± 18.66 | 46.15 ± 24.20 | <.0001 |
| FEV1/FVC | 0.81 ± 0.05 | 0.79 ± 0.06 | 0.48 ± 0.13 | <.0001 |
| Ave Exacerbations/Year, | NA | 0 [0, 3.6] | 0.3 [0, 4.4] | 0.0035 |
| Median [Min, Max] | | | | |
| Medication, n (%) | | | | |
| bagonist | 0 (0.00) | 1 (9.09) | 24 (60.00) | |
| bagonistlongact | 0 (0.00) | 0 (0.00) | 5 (12.50) | |
| CombcCSBagon | 0 (0.00) | 0 (0.00) | 22 (55.00) | |
| combivent | 0 (0.00) | 1 (9.09) | 12 (30.00) | |
| cortsterinhal | 0 (0.00) | 0 (0.00) | 7 (17.50) | |
| CortsterOral | 0 (0.00) | 0 (0.00) | 0 (0.00) | |
| ipratrop | 0 (0.00) | 0 (0.00) | 4 (10.00) | |
| MacAntibiotic | 0 (0.00) | 0 (0.00) | 1 (2.50) | |
| nebulizer | 0 (0.00) | 1 (9.09) | 19 (47.50) | |
| OsteoporosisMed | 2 (22.22) | 0 (0.00) | 5 (12.20) | |
| PhosphoInhibitor | 0 (0.00) | 0 (0.00) | 2 (5.00) | |
| tiotrop | 0 (0.00) | 1 (9.09) | 22 (55.00) | |

* Subjects may have missing information in some demographic variables.

TABLE 2

| | | | COPD | COPD | |
|---|---|---|---|---|---|
| | COPD | COPD | (GOLD | (GOLD | |
| | (GOLD I) | (GOLD II) | III) | IV) | |
| | (N = 4^{a}) | (N = 11^{a}) | (N = 12^{a}) | (N = 14^{a}) | p-Value |
| Age | 62.00 ± 13.24 | 67.91 ± 6.30 | 69.83 ± 7.40 | 68.79 ± 6.49 | 0.6911 |
| Gender (% Male) | 2 (50.00) | 7 (63.64) | 5 (41.67) | 6 (42.86) | 0.7241 |
| BMI | 34.50 ± 10.88 | 26.80 ± 5.82 | 63.63 ± 103.89 | 26.57 ± 10.58 | 0.3529 |
| Smoker (% Current) | 2 (50.00) | 2 (18.18) | 1 (8.33) | 0 (0.00) | 0.0289 |
| FEV1 (%) | 95.50 ± 17.75 | 63.91 ± 6.99 | 41.08 ± 5.30 | 22.43 ± 4.62 | <.0001 |
| FEV1/FVC | 0.60 ± 0.06 | 0.61 ± 0.05 | 0.50 ± 0.07 | 0.32 ± 0.04 | <.0001 |
| Ave Exacerbations/Year, | 0 [0, 0.2] | 0 [0, 2.8] | 0.7 [0, 4.4] | 1.05 [0, 3.5] | 0.0456 |
| Median [Min, Max] | | | | | |
| Medication, n (%) | | | | | |
| bagonist | 0 (0.00) | 5 (50.00) | 9 (75.00) | 9 (64.29) | |
| bagonistlongact | 0 (0.00) | 1 (10.00) | 2 (16.67) | 1 (7.14) | |
| CombcCSBagon | 1 (33.33) | 2 (20.00) | 8 (66.67) | 10 (71.43) | |
| combivent | 0 (0.00) | 3 (30.00) | 4 (33.33) | 4 (28.57) | |
| cortsterinhal | 0 (0.00) | 1 (10.00) | 2 (16.67) | 3 (21.43) | |
| CortsterOral | 0 (0.00) | 0 (0.00) | 0 (0.00) | 0 (0.00) | |
| ipratrop | 0 (0.00) | 1 (10.00) | 1 (8.33) | 1 (7.14) | |
| MacAntibiotic | 0 (0.00) | 0 (0.00) | 1 (8.33) | 0 (0.00) | |
| nebulizer | 0 (0.00) | 2 (20.00) | 4 (33.33) | 12 (85.71) | |
| OsteoporosisMed | 1 (25.00) | 1 (9.09) | 1 (8.33) | 2 (15.38) | |
| PhosphoInhibitor | 0 (0.00) | 0 (0.00) | 1 (8.33) | 1 (7.14) | |
| tiotrop | 1 (33.33) | 4 (40.00) | 6 (50.00) | 11 (78.57) | |

* Subjects may have missing information in some demographic variables.

### Flow Cytometry

Mouse ILCs were stained with antibodies against CD3, CD49b, IL-18Rα (eBiosciences), CD45, CD25, CD90, CD44 (Biolegend), and ST2 (MDBiosciences). Lineage cocktail included antibodies against TCRαβ, TCRγδ, CD5, CD27, F4/80, CD11c, Gr1, CD19, FCεRI, and B220 (eBiosciences). Intracellular antibodies included GATA-3, T-bet, IL-13, IL-5 and IFN-γ (eBiosciences). Live/dead fixable blue (Invitrogen) was used for all FACS experiments. For intracellular cytokine staining of ILCs, cells were incubated with indicated cytokines for indicated time periods, followed by 2-4 hour stimulation with PMA/Ionomycin and Brefeldin A before being surface stained, fixed, and permeabilized (FoxP3 staining kit, eBiosciences) for intracellular staining.

For staining of human ILCs (FIG. 8) from COPD patients, PBMC from nonsmoking healthy controls, smoking controls, or stable COPD patients were isolated from Heparin CP Tubes (BD Biosciences) and depleted of T and B cells using CD3 and CD19 microbeads (Miltenyi) according to manufacturer's protocol. All donors were drawn from the COPDgene pool and given informed consent according to study guidelines. Cells were then stained with antibodies against CD3, CD19, IL-7Rα, CD161 (eBiosciences), CRTH2, and CD56 (Biolegend). Lineage cocktail includes antibodies against TCRαβ, TCRγδ, CD34, CD14, CD16, CD1α, CD303a, CD123, FcεR1 (eBiosciences). Intracellular antibodies included GATA-3 and T-bet (eBiosciences). Live/dead fixable blue (Invitrogen) was used for all FACS experiments. All samples were run on an LSR II and analyzed using FlowJo.

### Adoptive Transfer of ILCs

For ILC2 transfers (FIGS. 4, 5, and 6), mice were treated intranasally on days 1, 3, and 5 with 2.5 μg of recombinant IL-33 to expand ILC2s, or with IL-12+ IL-18+IL-33 to expand ILC1s. On Day 7, CD45+ Viable CD3− CD49b− Lin− CD90+CD44+CD25+ST2+IL-18Rα− cells were purified and 1-1.5×10⁵ transferred via tail vein injections. In addition to ILC2s, 2.5-3×10⁶ lung-derived T/B/NK cells were transferred. Twelve hours later, recipient mice were infected with influenza A as described above. Analysis was done on days 7 and 10 post-infection. Cytokines in the BAL were measured by MSD at day 2 post-infection.

### Immunohistochemical Procedures

Identification of GFP-Positive Cells.

Paraffin-embedded lung sections were subjected to heat induced epitope retrieval (HIER) before immunohistochemical staining in an automated immunohistochemistry robot (AutostainerPlus, Dako). Briefly, sections were sequentially blocked with EnVision™ FLEX Peroxidase-Blocking Reagent and serum free protein block (both Dako) before incubation with a primary chicken anti-GFP antibody (Abcam). Next, sections were incubated with a goat anti-chicken antibody (Abcam) conjugated to HRP, followed by incubation with 3,3′-diaminobenzidine (DAB) substrate-chromogen solution, and counterstained with Mayer's hematoxylin (blue nuclei). Finally, sections were dehydrated through ethanol series, cleared in xylene, and mounted with Pertex (HistoLab).

Double Immunohistochemical Staining of GFP-Positive Cells and Influenza A

Immunoreactivity for GFP and influenza A were co-visualized in mouse lungs. Briefly, HIER treated sections were incubated with chicken anti-GFP antibodies, detected by goat anti-chicken antibodies (Abcam) conjugated to HRP, and a brown-colored immunoreaction product was produced using the peroxidase substrate DAB as chromogen. The sections were then treated with denaturing solution (Biocare Medical) and incubated with goat anti-influenza A antibodies (Abcam), followed by incubation with a HRP-conjugated rabbit anti-goat antibody (Dako). Finally, a green-colored influenza immunoreaction product was produced using the peroxidase substrate Vina Green as chromogen (Biocare Medical). Hematoxylin was used as background staining (blue nuclei), and the tissue sections were cleared in xylene and mounted with Pertex.

Triple Immunohistochemical Staining of GFP, GATA-3, and Influenza A

HIER treated tissue sections were incubated with goat anti-influenza A antibody, followed by incubation with a HRP-conjugated rabbit anti-goat antibody and development of the brown DAB chromogen at the site of influenza immunoreactivity. Next, sections were treated with denaturing blocking solution and incubated with chicken anti-GFP and rabbit anti-GATA3 primary antibodies (Abcam). This was followed by incubation with goat anti-chicken and goat anti-rabbit secondary antibodies conjugated to Alexa 488 or 555, respectively (Life Technologies). Finally, the tissue was treated with the DNA-binding fluorochrome Hoechst (blue nuclei), and mounted with PBS/glycerol. The immunoreactivity for all 3 markers was captured and digitized by a combined bright field and epifluoroscence digital slide scanner unit (Olympus VS120).

### In Situ Hybridization (ISH)

IL-12 and IL-18 mRNA were visualized using the RNAscope 2.0 FFPE assay kit, according to the manufacturer's instructions (Advanced Cell Diagnostics). Briefly, tissue sections were deparaffinized, incubated with endogenous enzyme block, boiled in pretreatment buffer, and treated with protease, followed by target probe hybridization using Mm-IL12b (319551, ACD) and Mm-IL18 (416731, ACD) probes. Probes against the housekeeping gene PPIB or the bacterial gene DapB served as positive and negative controls, respectively. The target RNA was then amplified and detected with DAB chromogen. Finally, the tissue sections were dehydrated and mounted using Pertex.

Combined Visualization of IL-12 or IL-18 mRNA and GFP-Positive ILCs

Tissue sections previously stained for IL-12 or IL-18 mRNA through in situ hybridization were subsequently incubated with a chicken anti-GFP antibody, followed by detection using a goat anti chicken-HRP and Vina Green chromogen development.

### Quadruple Histochemistry and Quantification of GATA-3 Intensity in GFP Cells

Sections were subjected to DNA/nuclear staining (DAPI, Alexa 355) combined with triple immunohistochemical staining for GFP, GATA-3 (immunofluoroscence with Alexa 488 and Alexa-555, respectively), and influenza (bright field visualization with DAB chromogen). All staining channels were digitized by a combined fluorescence and bright field Olympus VS-120 virtual slide microscope to generate one high-resolution image of the entire section for each marker. The intensity of the GATA-3 immunostaining in individual GFP-positive cells was measured using an automated region-of-interest (ROI) based method (software ImageJ 1.47v). Briefly, starting with the Alexa 488 image, intensity threshold was adjusted, locked, and used to create ROIs corresponding to GFP-positive cells. The multiple GFP ROIs were then pasted into the corresponding Alexa-555 image and used to measure the intensity of GATA-3 (i.e., Alexa555 mean intensity) in each GFP-positive ROI. Similarly, the GFP ROIs were pasted into the Alexa 355 image, and the intensity of DAPI in the same GFP-positive ROIs was calculated in order to adjust the GATA-3 intensity for nuclear content. The scanned bright field image of influenza staining was used to visualize the spatial relationship between GFP cells, GATA-3 intensity and regional ongoing infection.

Quantification of Influenza A in GFP+ Cells

Sections were subjected to immunohistochemical staining for GFP and influenza A (bright field visualization with DAB and Vina Green, respectively), and digitalized by Olympus VS-120 virtual slide microscope. Total immunoreactivity of influenza A and the total tissue area were calculated using computerized image analysis (Visiomorph-DP, Denmark). Influenza A positivity was calculated as the % of the total tissue area with influenza A immunoreactivity.

### Statistical Analysis

COPD Samples

The one-way mixed effect ANOVA model with heterogeneous-within-group variance was applied for comparisons of continuous measurements. The linear contrast was used to test linear trend between measurements and levels of average exacerbations per year. Fisher's exact test was used for comparison of categorical measurements, and Wilcoxon test was used to compare average exacerbations per year in the demographic tables. The Pearson correlation coefficient was used to evaluate correlation between the two continuous measurements. SAS 9.3 was used for the statistical analysis.

All Other Analyses

Data was analyzed and expressed as stated in the Brief Description of the Drawings, using GraphPad Prism. Error bars represent Standard Error of the Mean.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance. The present invention is further described by the following claims.

1. A method of inhibiting the conversion of innate lymphoid cells (subset 2) (ILC2s) into innate lymphoid cells (subset 1) (ILC1s) comprising contacting said ILC2s with a modulator that prevents the switch of said ILC2s to T-bet+ IFNγ+ ILC1s, maintains or suppresses the level of IL-12 receptor and/or IL-18 receptor expression in ILC1s, and/or maintains or increases the level of ST2, CRTH2 and/or GATA3 expression in ILC2s.

2. The method of claim 1, wherein the modulator is an inhibitor or an agonist.

3. The method of claim 2, wherein the inhibitor or agonist, is a small molecule or an antibody or antigen binding fragment thereof.

4. The method of claim 3, wherein the antibody or antigen-binding fragment thereof is selected from a human antibody, a humanized antibody, a chimeric antibody, a recombinant antibody, a bi-specific antibody, a multi-specific antibody, and an antigen-binding fragment thereof.

5. The method of any one of claims 1 to 4, wherein the modulator is a monoclonal antibody or an antigen binding fragment thereof.

6. The method of claim 5, wherein the monoclonal antibody or antigen binding fragment thereof is selected from an Fv, an Fab, an F(ab′)2, an Fab′, a dsFv fragment, a single chain Fv (scFV), an sc(Fv)2, a disulfide-linked (dsFv), a diabody, a triabody, a tetrabody, a minibody, or a single chain antibody.

7. The method of any one of claims 1-6, wherein said ILC2s contacted with said modulator are localized to the lung or to lung tissue.

8. The method of any one of claims 1-6, wherein said ILC2s contacted with said modulator are circulating ILC2s.

9. A method of preventing or treating a disease or disorder associated with lung inflammation in a subject in need thereof, comprising administering to said subject a disease-modifying medication, whereby said subject is determined to have elevated levels of ILC in one or more samples taken from said subject compared to a predetermined level of ILC1s and/or compared to the levels of ILC1s in one or more control samples.

10. A method of preventing or treating a disease or disorder associated with lung inflammation in a subject in need thereof, comprising administering to said subject a disease-modifying medication, whereby said subject is determined to have an elevated ratio of ILC1s/ILC2s in one or more samples taken from said subject compared to a predetermined ratio of ILC1s/ILC2s and/or compared to the ratio of ILC1s/ILC2s in one or more control samples.

11. A method of preventing or treating exacerbation of a disease or disorder associated with lung inflammation in a subject in need thereof, comprising administering to said subject a disease-modifying medication, whereby said subject is determined to have elevated levels of ILC1s in one or more samples taken from said subject compared to a predetermined level of ILC1s and/or compared to the levels of ILC1s in one or more control samples.

12. The method of any one of claims 9-11, wherein said lung inflammation is caused by cigarette smoke, bacterial infection, or viral infection.

13. The method of any one of claims 9-12, wherein said disease or disorder is chronic obstructive pulmonary disease (COPD).

14. The method of claim 11, wherein said exacerbation is caused by cigarette smoke, bacterial infection, or viral infection.

15. The method of any one of claims 9-14, wherein said lung inflammation or exacerbation of said disease or disorder is caused by viral infection.

16. The method of any one of claims 9-15, further comprising inhibiting the migration of ILC1s to areas associated with viral replication.

17. A method of selecting a patient diagnosed with a disease or disorder associated with lung inflammation as a candidate for treatment with disease-modifying medications, comprising selecting the patient for treatment if the patient is determined to have an elevated ratio of ILC1s/ILC2s in one or more samples taken from said subject compared to a predetermined ratio of ILC1s/ILC2s and/or compared to the ratio of ILC1s/ILC2s in one or more control samples.

18. The method of claim 17, wherein the disease-modifying medication is a modulator of ILC2 to ILC1 conversion.

19. The method of claim 17, wherein the disease-modifying medication is a bronchodilator, an inhaled steroid, a combination inhaler, an oral steroid, or a phosphodiesterase-4 inhibitor.

20. The method of claim 17, wherein the modulator is an inhibitor or an agonist.

21. The method of claim 20, wherein the inhibitor or agonist is a small molecule compound or an antibody or antigen binding fragment thereof.

22. The method of claim 21, wherein the antibody or antigen-binding fragment thereof is selected from a human antibody, a humanized antibody, a chimeric antibody, a recombinant antibody, a bi-specific antibody, a multi-specific antibody, and an antigen-binding fragment thereof.

23. The method of any one of claims 20-22, wherein the modulator is a monoclonal antibody or an antigen binding fragment thereof.

24. The method of claim 23, wherein the monoclonal antibody or antigen binding fragment thereof is selected from an Fv, an Fab, an F(ab′)2, an Fab′, a dsFv fragment, a single chain Fv (scFV), an sc(Fv)2, a disulfide-linked (dsFv), a diabody, a triabody, a tetrabody, a minibody, or a single chain antibody.

25. The method of any one of claims 9-24, wherein said determination of having an elevated ratio of ILC1s/ILC2s in one or more samples taken from said subject compared to a predetermined ratio of ILC1s/ILC2s and/or compared to the ratio of ILC1s/ILC2s in one or more control samples, or said determination of elevated levels of ILC1s in one or more samples taken from said subject compared to a predetermined level of ILC1s and/or compared to the levels of ILC1s in one or more control sample, is based on a determination of a switch between a molecular signature of ILC2 to a molecular signature of ILC1.

26. The method of claim 25, wherein said molecular signature of ILC2 corresponds to the expression of one or more Th2-associated transcripts selected from the group consisting of Gata3, Rora, Il4, Il5, Il9, Il13, Penk (proenkephalin), Areg (amphiregulin), Il17rb, and Il1rl1.

27. The method of claim 25 or 26, wherein said molecular signature of ILC1 corresponds to a lower expression of one or more Th2-associated transcripts according to claim 26 and higher levels of one or more transcripts selected from the group consisting of Tbx21, Ifng, Il12rb2, Il18r1, Cxcr3, and Ccr5, wherein said levels are compared to the level of said transcripts in ILC2.
